(19) United States
(12) Patent Application Publication
(10) Pub. No.: US 2024/0043473 A1
(43) Pub. Date: Feb. 8, 2024

(54) NOOTROPIC PEPTIDES FOR TREATING LYSOSOMAL STORAGE DISEASES

(71) Applicant: Phoenix Nest Inc., Brooklyn, NY (US)

(72) Inventors: Jill WOOD, Brooklyn, NY (US); Alexey PSHEZHETSKY, Quebec (CA); Poulomee BOSE, Quebec (CA)

(21) Appl. No.: 18/264,542

(22) PCT Filed: Feb. 9, 2022
(86) PCT No.: PCT/US2022/015818

§ 371 Date: Aug. 7, 2023

Related U.S. Application Data
(60) Provisional application No. 63/147,509, filed on Feb. 9, 2021.

(51) Int. Cl. C07K 7/06; A61P 3/00; A61K 9/00

(52) U.S. Cl. CPC C07K 7/06; A61P 3/00; A61K 9/0043

(57) ABSTRACT

Provided are compositions and methods for treating progressive neurological childhood symptoms and conditions associated with lysosomal storage disorders (LSD) such as neurological mucopolysaccharidoses. The compositions may include nootropic peptides such as Semax, which can be N-terminally acetylated and/or C-terminally amidated. The peptides can be effectively delivered by intranasal administration into the brain parenchyma, where they exert a neuroprotective and anti-inflammatory effect and delay or restore neuropathophysiological defects such as neuropsychiatric problems, developmental delays, mental retardation and dementia.

### CROSS REFERENCE TO RELATED APPLICATIONS

This application claims the benefit under 35 U.S.C. § 119(e) of the U.S. Provisional Application Ser. No. 63/147,509, filed Feb. 9, 2021, the content of which is hereby incorporated by reference in its entirety.

### BACKGROUND

Lysosomal storage disorders (LSD) are a group of rare inherited metabolic disorders that result from defects in lysosomal function. The lysosome processes unwanted materials into other substances that the cell can utilize. Lysosomes break down this unwanted matter via enzymes, highly specialized proteins essential for survival. The lysosomal dysfunction usually is a consequence of deficiency of a single enzyme required for the metabolism of lipids, glycoproteins (mucopolysaccharides) or glycosaminoglycans. The lysosomal dysfunction can also be caused by defects of a protein involved in the transport of metabolites, or lysosomes or proteins essential for production and functioning of lysosomes. Individually, LSDs occur with incidences of less than 1:100,000; however, as a group, the incidence is about 1:5,000-1:10,000. Most of these disorders are autosomal recessively inherited but a few are X-linked recessively inherited.

A major subtype of LSD is mucopolysaccharidoses (MPS), caused by the absence or malfunctioning of lysosomal enzymes needed to break down glycosaminoglycans (GAGs). These long chains of sugar carbohydrates occur within the cells that help build bone, cartilage, tendons, corneas, skin and connective tissue. GAGs (formerly called mucopolysaccharides) are also found in the fluids that lubricate joints.

Individuals with MPS either do not produce enough of one of the eleven enzymes required to break down these sugar chains into simpler molecules, or they produce enzymes that do not work properly. Over time, these GAGs collect in the cells, blood and connective tissues. The result is permanent, progressive cellular damage which affects appearance, physical abilities, organ and system functioning. Most MPS affect the central nervous system of children and result in severe progressive neurodegenerative decline eventually leading to handicap and death.

No effective therapies for neurological LSD are available yet. Enzyme replacement therapies (ERT) are targeted to peripheral pathology due to inability of the recombinant enzyme to enter the brain. ERTs, however, are ineffective for the neurological forms due to the enzyme's inability to cross the blood-brain barrier (BBB). In MPS IIIC and other diseases, caused by defects in transmembrane proteins, an additional constraint is the absence of cross-correction between the cells. Delivering replacement enzyme intrathecally, thus bypassing the BBB, is difficult to implement clinically due to the invasive nature of the procedure.

Furthermore, intrathecal delivery is not possible for membrane enzymes and proteins. Haemopoietic stem cell transplant is the only effective therapeutic approach for a group of few neuropathic LSDs, where the missing enzyme is soluble and can be effectively secreted by donor cells.

Direct delivery of a gene therapy vector to the brain has shown good efficacy in mice and dogs, but diffusion of the AAV vector, commonly used in these protocols, is limited to <0.5 cm³ around the injection site. Besides, potential immunological problems and the long-term consequences of stereotaxic injection of AAV viruses are not well studied. Also, the risks of immunological responses and the long-term consequences of stereotaxic injection of AAV viruses are not established. Several novel therapies are currently emerging, including genome editing using CRISPR-CAS or ZFN (zinc finger nuclease) technologies or ERT with BBB-penetrating enzymes, where therapeutic enzymes are linked with monoclonal antibodies to insulin or transferrin receptors that successfully target their ligands to the brain parenchyma. Outcomes of clinical trials for these strategies are either still unknown or failed to produce desired effects. Thus, currently there is no effective treatment for neurological LSDs caused by defects in membrane proteins.

### SUMMARY

The instant disclosure, in various embodiments, provides therapies for progressive neurological childhood symptoms and conditions associated with lysosomal storage disorders (LSD) using nootropic peptides. Such peptides can be effectively delivered by intranasal administration into the brain parenchyma, where they exert a neuroprotective effect and delay or restore neuropathophysiological defects such as neuropsychiatric problems, developmental delays, mental retardation and dementia.

One embodiment provides a method for treating a neuropathophysiological condition in a patient in need thereof, comprising administering to the patient an effective amount of an agent that increases the biological activity or physiological levels of brain-derived neurotrophic factor (BDNF). In some embodiments, the patient suffers from a lysosomal storage disorder (LSD). In some embodiments, the LSD is selected from the group consisting of a lipid storage disorder, a mucopolysaccharidosis, a glycoprotein storage disorder, and a mucolipidosis. In some embodiments, the LSD is a neurological mucopolysaccharidosis (MPS), such as MPS I, MPS II, MPS III, MPS VII, and MPS IX.

In some embodiments, the neuropathophysiological condition is selected from the group consisting of dementia, aggressive behavior, hyperactivity, seizure, deafness and loss of sleep and vision.

In some embodiments, the agent is a peptide that comprises the amino acid sequence of MEHFPGP (SEQ ID NO:1) or an analog thereof. In some embodiments, the analog comprises an amino acid sequence selected from the group consisting of MGHFPGP (SEQ ID NO:3), MEHFXPGP (SEQ ID NO:4), MGHFXPGP (SEQ ID NO:5), MEHFPAP (SEQ ID NO:6), MEHFXPAP (SEQ ID NO:7), and MGHFXPAP (SEQ ID NO:8), wherein X represents any amino acid residue. In some embodiments, the peptide is N-terminal acetylated and/or C-terminal amidated.

In some embodiments, the administering is intranasal.

Also provided, is a method for treating a mucopolysaccharidosis (MPS), in particular MPS III (e.g., MPS IIIC) in a patient in need thereof, comprising intranasal administration to the patient an effective amount of a peptide that comprises an amino acid sequence selected from the group consisting of MEHFPGP (SEQ ID NO:1), MGHFPGP (SEQ ID NO:3), MEHFXPGP (SEQ ID NO:4), MGHFXPGP (SEQ ID NO:5), MEHFPAP (SEQ ID NO:6), MEHFXPAP (SEQ ID NO:7), and MGHFXPAP (SEQ ID NO:8), wherein X represents any amino acid residue. In some embodiments, the peptide's N-terminus is acetylated and/or C-terminus is amidated.

Also provided, in another embodiment, is a formulation for intranasal administration, comprising an agent that increases the biological activity of brain-derived neurotrophic factor (BDNF).

### BRIEF DESCRIPTION OF THE DRAWINGS

FIG. 1 shows that application of AVP6 on hippocampal slices of MPSIIIC mice restored amplitude and frequency of miniature excitatory postsynaptic currents.

FIG. 2 shows that intranasal administration of AVP6 restored BDNF levels in MPSIIIC

mice.

FIG. 3 shows that intranasal administration of AVP6 significantly recovered memory deficits in MPSIIIC mice.

FIG. 4 shows the effects of AVP6 on synaptic neurotransmission.

FIG. 5 shows the effects of AVP6 on evoked synaptic events.

FIGS. 6 and 7 show the effects of AVP6 on synaptic morphology and synaptic proteins in cultured MPSIIIC mouse neurons.

FIG. 8 shows that AVP6 reduces hyperactivity and rescues reduced anxiety in MPSIIIC mice.

FIG. 9 shows that AVP6 recovers reduced fear in MPSIIIC mice.

FIG. 10-11 show the results of characterization of hippocampal slices of MPSIIIC mice.

FIG. 12-14 show the results of characterization of iPSCs from skin fibroblasts of MPS IIIC patients.

FIG. 15 shows that AVP6 (ACTH₍₄₋₇₎PGP) rescues reduced amplitude and frequency of mEPSC in Hgsnat-Geo and Hgsnat^{P304L} MPS IIIC mice. Significant decrease in the amplitude (A, C) and frequency (B, D) of mEPSC recorded in CA1 pyramidal neurons in acute hippocampal slices from Hgsnat-Geo and Hgsnat^{P304L} MPS IIIC mice at the ages of P14-20 and P45-60 is observed as compared to age-matched WT controls. The deficit is rescued by bath application of AVP6 in the final concentration of 10 μM. The drug does not increase reduced frequency (E) and amplitude (F) of iEPSCs recorded in CA1 pyramidal neurons in acute hippocampal slices in Hgsnat-Geo MPS IIIC mice at the age of P14-20. All graphs show individual data, means and SD of experiments. Number of studied animals is shown at the panels. P values were calculated using Kruskal-Wallis test with Dunns post-hoc test.

FIG. 16 shows that AVP6 (ACTH₍₄₋₇₎PGP) rescues presynaptic deficits in Hgsnat-Geo and Hgsnat^{P304L} mice. (A) Representative current traces showing the amplitude of AMPA EPSCs in brain slices from WT, Hgsnat^{P304L} mice, and in brain slices from Hgsnat^{P304L} mice treated with 10 μM AVP6. (B-C) Significant decreases in the AMPA (B) and NMDA (C) ratios are observed in slices from Hgsnat^{P304L} mice as compared with the WT controls with the same intensity of stimulation (0.1 ms; 3 to 6 V cathodal pulses), but not in AVP6-treated brain slices from Hgsnat^{P304L} mice. (D-F) Decreased amplitude of PPR with interstimulus intervals of 50 ms, 100 ms. 200 ms, and 300 ms in hippocampal slices from Hgsnat-Geo and Hgsnat^{P304L} mice is restored by treatment with AVP6. Graphs show individual data, means and SD (B, C) or mean values and SD (D). Number of mice studied is shown in the graphs. P values were calculated using Kruskal-Wallis with Tukey's multiple comparison post-hoc test (B,C) or two-way ANOVA with Tukey's multiple comparison test (D-F). ****, *** and ** indicate a significant difference (p<0.0001, 0.001, and 0.01, respectively) between the WT and the untreated Hgsnat-Geo or Hgsnat^{P304L} mice. \hat{ }\hat{ }\hat{ }\hat{ }, \hat{ }\hat{ }, \hat{ } indicate a significant difference (p<0.0001, 0.01, and 0.05, respectively) between the untreated Hgsnat-Geo or Hgsnat^{P304L} mice and AVP6-treated Hgsnat-Geo or Hgsnat^{P304L} mice.

FIG. 17 shows that AVP6 (ACTH₍₄₋₇₎PGP) increases reduced levels of glutamatergic synaptic protein markers and BDNF in cultured neurons from Hgsnat^{P304L} MPS IIIC mice and in iPSC-derived cultured cortical neurons from human MPS IIIA and MPS IIIC patients. Immunocytochemical staining was conducted in cultured primary hippocampal neurons of Hgsnat^{P304L} mice (A) and iPSC-derived neurons of MPS IIIC (B, D) and MPS IIIA (C, D) patients for an axonal marker, NF-M and a synaptic marker, SYN1, a dendritic marker, MAP2, and BDNF or a glutamatergic presynaptic marker, VGLUT1, and a glutamatergic post-synaptic marker, PSD-95. Neurons from Hgsnat^{P304L} mice and iPSC-derived neurons of MPS IIIA and MPS IIIC patients show significantly reduced levels of VGLUT1+, PSD-95+, SYN1+ and BDNF+ puncta as compared with their respective controls. Levels of all four markers are significantly increased in the neurons cultured in the presence of 10 μM AVP6. Panels show representative images of stained neurons. Inserts show enlarged images of dendrites or axons taken at a distance of 10 μm from the soma. Bar graph equals 10 μm. Graphs show quantification of VGLUT+, PSD-95+, SYN1+ or BDNF+ puncta by ImageJ software. Individual values, means and SD from 8-10 cells in each group are shown. P values were calculated using ANOVA with Tukey post-hoc test.

FIG. 18 shows that short-term treatment with AVP6 (ACTH₍₄₋₇₎PGP) partially rescues neurobehavior manifestations and increases hippocampal BDNF levels in symptomatic MPS IIIC mice. (A and B) Hgsnat^{P304L} mice at the ages of 4 and 6 months show significant increase in the time spend in the central zone (A) and total distance traveled (B) in the Open Field test as compared with age-matched WT controls consistent with reduced anxiety and hyperactivity. Both parameters are normalized in the mice, intranasally administered with AVP6 at a dose of 50 μg/kg BW 17 h prior to the behavioral analysis. Low dose (LD, 10 μg/kg BW) and high dose (HD, 500 μg/kg BW) of the peptide do not rescue hyperactivity in the Open Field test. (C and D) Four-month-old Hgsnat^{P304L} mice show significant increase in the percent of time spent in open arms and in the number of open arm entries in the Elevated Plus Maze test, as compared with age-matched WT controls. Both parameters are normalized in mice, intranasally administered with AVP6 at a dose of 50 μg/kg BW 17 h prior to the behavioral analysis. (E and F) A significant decrease in discrimination index and recognition index in the Novel Object Recognition test is observed in 4-month-old Hgsnat-Geo mice as compared to age-matched WT controls indicating deficit of short-term memory. This deficit is rescued in Hgsnat-Geo mice daily treated by intranasal administration of AVP6 at a dose of 50 μg/kg BW for 10 consecutive days preceding the analysis. (G) Mature BDNF levels are reduced in the hippocampi of saline-treated 4-month-old Hgsnat-Geo mice as compared with WT mice, and are partially rescued by 10-day treatment with AVP6 at a dose of 50 μg/kg BW. All graphs show individual data, means and SD. P-values were calculated using ANOVA with Tukey's multiple comparisons test. Number of animals studied is shown in the graphs.

FIG. 19 presents LC-MS/MS MRM analysis that shows effective delivery of AVP6 (ACTH₍₄₋₇₎PGP) to the brain after intranasal administration. WT C57B16 4-month-old male mouse was dosed intranasally with 10 μl of 50 mM AVP6 in saline (5 μl/nostril). One hour after dosing, the mouse was anesthetized with sodium pentobarbital, and 500 μl of blood collected by cardiac puncture. Mouse was then sacrificed by cranial dislodgement and its brain and visceral organs extracted. The brain was dissected into 4 segments (frontal to dorsal) as shown in the figure. Tissues and blood plasma were homogenized in acetonitrile (1:4, tissue/solvent ratio). The extracts were spiked with heavy isotope-labelled (Phe U-¹³C₉; U-¹⁵N) AVP6 peptide as an internal standard, and analyzed by targeted LC-MS/MS, using parallel reaction monitoring on Orbitrap Exploris 480 instrument. The concentration of the peptide in the brain (2.8-0.9 fmol/μg) is higher than in blood plasma or visceral organs and exceeds the concentration estimated to be effective in restoring the neurotransmission in electrophysiological experiments.

FIG. 20 shows Hgsnat^{P304L} mice treated with AVP6 (ACTH₍₄₋₇₎PGP) reveal delay in development of neurobehavioral abnormalities at the age of 4 months. Vehicle (saline)-treated Hgsnat^{P304L} male and female mice at the age of 4 months show hyperactivity (increased total distance traveled in the Open Field test, A), reduced anxiety/fear (increased time spend in the central zone in the Open Field test, B, and increased number of open arm entries in the Elevated Plus Maze test C), deficits in spatial/short-term memory (reduced alterations between arms in the Y-Maze test, D, decrease in the discrimination, E, and recognition, F, index in the Novel Object Recognition test). Hgsnat^{P304L} male and female mice, treated daily, starting from the age of 3 weeks with AVP6 (50 μg/kg BW), show the rescue of all above deficits. Individual results, means and SD from experiments performed with 12 or more mice per genotype, per treatment are shown. P values were calculated using one-way ANOVA with Tukey post-hoc test.

FIG. 21 shows Hgsnat^{P304L} mice treated with AVP6 (ACTH₍₄₋₇₎PGP) reveal partial rescue of synaptic protein markers and neuroinflammation at the age of 5 months. Deficient levels of protein markers of glutamatergic synaptic neurotransmission, VGUT1 and PSD-95 (A) and BDNF (B) are rescued, and increased levels of activated CD68+ microglia and GFAP+ astrocytes are reduced in the somatosensory cortex and hippocampus of Hgsnat^{P304L} mice, treated daily with AVP6 (50 μg/kg BW) starting from the age of 3 weeks. Panels show representative images of brain cortex (layers 4-5) and CA1 area of the hippocampus of 5-month-old WT, and Hgsnat^{P304L} mice, treated or not with AVP6. The tissues are stained with antibodies against PSD-95 (red) and VGLUT1 (green) (A), BDNF (red) and MAP2 (green) (B), GFAP (green) and NeuN (red) (C), and CD68 (green) and NeuN (red) (D). In all panels DAPI (blue) was used as a nuclear counterstain. Scale bar equals 25 μm. The graphs show quantification of fluorescence with ImageJ software. Individual results, means and SD from experiments performed with 3 mice per genotype (3 areas/mouse), per treatment are shown. P values were calculated using ANOVA with Tukey post-hoc test.

FIG. 22 shows that Hgsnat^{P304L} mice treated with AVP6 (ACTH₍₄₋₇₎PGP) reveal delay in development of neurobehavioral abnormalities at the age of 6-7 months. Hgsnat^{P304L} mice treated with the vehicle (saline) at the age of 6 months show hyperactivity (increased total distance traveled, A) and reduced anxiety/fear (increased time spend in the central zone, B) in the Open Field test. They also demonstrate deficits in spatial/short-term memory (reduced alterations between arms, C) in the Y-Maze test. Hgsnat^{P304L} mice, treated daily with AVP6 (50 μg/kg BW), starting from the age of 3 weeks, show rescue of all above deficits. Individual results, means and SD are shown. P values were calculated using one-way ANOVA with Tukey post-hoc test.

FIG. 23 shows that chronic daily treatment with AVP6 (ACTH₍₄₋₇₎PGP) increases survival and reduces splenomegaly in Hgsnat^{P304L} mice. (A) Kaplan-Meier plot showing survival of saline-treated Hgsnat^{P304L} (n=8) and AVP6-treated Hgsnat^{P304L} male and female mice (n=9), and their saline-treated (n=8) and AVP6-treated (n=7) WT counterparts. The significance of survival rate differences between strains was determined by the Mantel-Cox test (P<0.05). By the age of 43 weeks, all saline-treated Hgsnat^{P304L} mice had to be euthanized on the veterinarian request due to urinary retention, while AVP6-treated Hgsnat^{P304L} mice survived to the average age of 49 weeks. (B) Wet organ weight of treated and untreated Hgsnat^{P304L} and WT mice at sacrifice (9.5-11 months). Enlargement of spleen as compared with age-matched WT controls, consistent with the lysosomal storage and inflammatory cell infiltration, is detected in saline-treated Hgsnat^{P304L} but not in AVP6-treated Hgsnat^{P304L} mice. Graphs shows individual data, means and SD. P values were calculated using ANOVA with Tukey post-hoc test.

FIG. 24 shows that Hgsnat^{P304L} mice treated with AVP6 (ACTH₍₄₋₇₎PGP) reveal partial rescue of synaptic protein markers and neuroinflammation at the age of 10-11 months. Deficient levels of protein markers of glutamatergic synaptic neurotransmission, VGUT1 and PSD-95 (A), BDNF (B) and SYN1 (C) are rescued in hippocampus and partially rescued in the somatosensory cortex of Hgsnat^{P304L} mice, treated daily with AVP6 (50 μg/kg BW) starting from the age of 3 weeks. Treatment also reduces levels of activated GFAP+ astrocytes in the cortex (D) and CD68+ microglia in both cortex and hippocampus (E). Panels show representative images of brain cortex (layers 4-5) and CA1 area of the hippocampus of 5-month-old WT mice daily treated with saline and Hgsnat^{P304L} mice, treated with saline or AVP6. The tissues are stained with antibodies against PSD-95 (red) and VGLUT1 (green) (G), BDNF (red) and MAP2 (green) (H), GFAP (green) and NeuN (red) (I) and CD68 (green) and NeuN (red) (J). In all panels DAPI (blue) was used as a nuclear counterstain. Scale bar equals 25 μm. The graphs show quantification of fluorescence with ImageJ software. Individual results, means and SD from experiments performed with 3 mice per genotype (3 areas/mouse), per treatment are shown. P values were calculated using ANOVA with Tukey post-hoc test.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “peptide” or “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Treatment for Lysosomal Storage Disorders (LSD)

It is discovered herein that the expression of BDNF (Brain Derived Neurotrophic Factor) was significantly reduced in two animal (mouse) models of Mucopolysaccharidosis BIC (MPS IIIC or Sanfilippo disease C). More specifically, it was observed that the levels of the mature BNDF were decreased. Likewise, BDNF levels also decreased in cultured human iPSC (induced pluripotent cells)-derived neurons of MPS IIIA and MPS IIIC patients

Treatment with AVP6 (N-terminally acetylated Semax, a nootropic peptide having the amino acid sequence of MEHFPGP (SEQ ID NO:1)) was able to restore the expression of BDNF in mouse brain and in MPS IIIC mouse and MPS IIIC and MPS IIIA human cultured neurons. Meanwhile, AVP6 also rescued reduced levels of Synapsin 1, PSD-95 and VGLUT1 levels in mouse brains and neuronal cultures. As shown in the examples, these synaptic markers (including Synapsin 1, Synaptophysin, PSD-59, VGLUT1, Gephyrin, VGAT) are deficient in the mouse models of MPS IIIC and cultured human iPSC (induced pluripotent cells)-derived neurons of MPS IIIA and MPS IIIC patients.

Treatment with AVP6 also increased the life-span of MPS BIC mice and reduced neuronal pathology, including astrogliosis, microgliosis, levels of inflammatory cytokines in the brain at 5 and 10-11 months of age as well as splenomegaly at 10-11 months of age.

Also important, at the behavioural level, AVP6 treatment reduced anxiety and fear deficit and hyperactivity, and improved working (short-term and spatial) memory. Such benefits of the AVP6, it is contemplated, are also applicable to other lysosomal storage disorders (LSD) which are associated with similar neuropathophysiological conditions. In addition, agents besides AVP6, more broadly referred to as nootropic agents, are believed to be effective as well.

In accordance with one embodiment of the present disclosure, therefore, provided is a method for treating a neuropathophysiological condition in a patient in need thereof, comprising administering to the patient an effective amount of an agent that increases synaptic transmission.

In another embodiment, the present disclosure provides a method for treating a neuropathophysiological condition in a patient in need thereof, comprising administering to the patient an effective amount of an agent that increases the biological activity of brain-derived neurotrophic factor (BDNF).

Examples of agents that can increase synaptic transmission or the biological activity (expression or activity) of BDNF include nootropic agents. Nootropic agents are agents that can improve cognitive function, particularly executive functions, memory, creativity, or motivation, in individuals.

An example group of nootropic agents are CNS stimulants, such as amphetamine, methylphenidate, eugeroics (armodafinil and modafinil), caffeine, and nicotine. Another example group include racetams, such as fasoracetam, nebracetam, nefiracetam, levetiracetam or other members of the racetam family of compounds including pharmaceutically acceptable salts and solvates thereof.

Yet another group of nootropic agents are cholinergics, such as citicoline, choline bitartrate, and alpha-GPC (L-Alpha glycerylphosphorylcholine). Other examples include tolcapone, levodopa, atomoxetine, desipramine, nicergoline and ISRIB (integrated stress response inhibitor, or trans-N,N′-(Cyclohexane-1,4-diyl)bis(2-(4-chlorophenoxy)acetamide)).

Still, further examples include Acetyl L-Carnitine (ALCAR), Alpha-GPC, Alpha-Lipoic Acid (ALA), Aniracetam, Ashwagandha, Artichoke Extract (Luteolin), Bacopa Monnieri, Berberine, Black Seed Oil, Cacao, Caffeine, Cat's Claw, CBD Oil, Choline, Choline Bitartrate, Choline Citrate, Citicoline (see CDP-Choline), CDP-Choline, Centrophenoxine. Coconut & MCT Oil, Coluracetam, CoQ10 & Ubiquinol, Creatine, DHA (Omega 3). DHEA, DMAE, 5-HTP, Forskolin (Coleus root), GAB A, Ginkgo Biloba, Ginseng, Gotu Kola, Glycine, Holy Basil (Tulsi), Huperzine-A, Iodine, Kava Kava, Kratom, Lion's Mane, L-Carnosine, L-Dopa (Mucuna Pruriens), Lemon Balm, L-Glutamine, Lithium Orotate, L-Theanine, Maca, Magnesium, Medicinal Mushrooms, Methylene Blue, Melatonin, N-Acetyl L-Cysteine, N-Acetyl L-Tyrosine, NADH, Nefiracetam, Nicotine, Noopept, Oat Straw, Oxiracetam, Phenibut, Phenylpiracetam, Picamilon, Pine Bark Extract (Pycnogenol®), Piperine, Piracetam, Rhodiola Rosea, Phenylalanine, Phenylethylamine (PEA), Phosphatidylcholine (PC), Phosphatidylserine (PS), PQQ, Pramiracetam, Pterostilbene, Quercetin, Resveratrol, Rosemary, Saffron, SAM-e, St John's wort, Sulbutiamine, Taurine, Tryptophan, Turmeric, Tyrosine, Uridine Monophosphate, Valerian, Vinpocetine, Vitamin B1 (Thiamine), Vitamin B3 (Niacin), Vitamin B5 (Pantothenic Acid), Vitamin B6 (Pyridoxine), Vitamin B8 (Inositol), Vitamin B9 (Folate), Vitamin B12 (Cobalamin), Vitamin D, and Zinc.

Yet other examples of nootropic peptides are AVP6 and its analogs. AVP6 is N-terminally acetylated Semax. Semax is a drug used for a broad range of conditions but predominantly for its purported nootropic, neuroprotective, and neurorestorative properties. In animals, Semax rapidly elevates the levels and expression of brain-derived neurotrophic factor (BDNF) and its signaling receptor TrkB in the hippocampus, and rapidly activates serotonergic and dopaminergic brain systems.

Semax is a synthetic analogue of adrenocorticotropic hormone 4-10 hexapeptide (ACTH₍₄₋₁₀₎), consisting of an ACTH fragment, ACTH₍₄₋₇₎ and the C-terminal tripeptide Pro-Gly-Pro, (ACTH₍₄₋₇₎PGP; MEHFPGP (SEQ ID NO: 1)). ACTH (NP_000930.1) includes three ACTH domains (underlined in Table 1 below).

TABLE 1
Human ACTH Sequence (SEQ ID NO: 2)

```
    1 MPRSCCSRSG ALLLALLLQA SMEVRGWCLE
        SSQCQDLITE SNLLECIRAC KPDLSAETPM
  61 FPGNGDEQPL TENPRKYVMG HERWDRFGRR
        NSSSSGSSGA GQKREDVSAG EDCGPLPEGG
121 PEPRSDGAKP GPREGKRSYS MEHFRWGKPV
        GKKRRPVKVY PNGAEDESAE AFPLEFKREL
181 TGQRLREGDG PDGPADDGAG AQADLEHSLL
        VAAEKKDEGP YRMEHFRWGS PPKDKRYGGE
241 MTSEKSQTPL VTLFKNAIIK NAYKKGE
```

The core residues within these ACTH domains are MGHF (residues 79-82 of SEQ ID NO:2) and MEHF (residues 141-144 or 223-226 of SEQ ID NO:2). Semax is a fusion between one of these strings (MEHF, residues 141-144 or 223-226 of SEQ ID NO:2) with PGP. An example analog of Semax can use the other core sequence (MGHF, residues 79-82 of SEQ ID NO:2) as well. In some embodiment, one, two or three amino acid residues may be inserted before PGP. In some embodiments, the PGP tripeptide may be replaced by PAP where A is analogous to G.

Accordingly, Semax has the following example analogs. In some embodiments, the Semax or the analog is N-terminally acetylated which is herein shown to increase the stability of the peptide. In some embodiments, the Semax or the analog is C-terminally amydated which is herein shown to increase the stability of the peptide.

TABLE 2
Semax Analogs

| Analogs | SEQ ID NO: |
|---|---|
| MGHFPGP | 3 |
| MEHFXPGP | 4 |
| MGHFXPGP | 5 |
| MEHFPAP | 6 |
| MEHFXPAP | 7 |
| MGHFXPAP | 8 |

X: any amino acid residue

Additional analogs can also be created and tested. In one embodiment, the analog includes one, two or three addition, deletion, substitution or the combinations thereof from SEQ ID NO:1.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

TABLE 3
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

TABLE 4
Conservative Amino Acid Substitutions

| For Amino Acid | Substitution With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, L-Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

In some embodiments, the peptide has a length that is not longer than 50, 40, 30, 20, 15, 10, 9, 8, or 7 amino acid residues.

The agents here are able to increase the expression and/or activity of BDNF in patients where the BDNF activity/expression is reduced. In some embodiments, the patient has a lysosomal storage disease (LSD).

Lysosomal storage diseases (LSDs) are a group of about 50 rare inherited metabolic disorders that result from defects in lysosomal function. Lysosomes cytoplasmic organdies containing hydrolytic enzymes that digest large molecules and pass the fragments onto other parts of the cell for recycling. This process requires several critical enzymes. Most of these disorders are autosomal recessively inherited such as Niemann-Pick disease, type C, but a few are X-linked recessively inherited, such as Fabry disease and Hunter syndrome (MPS II).

The LSDs are generally classified by the nature of the primary stored material involved, and can be broadly broken into the following, (A) Lipid storage disorders, including sphingolipidoses, including Gaucher's and Niemann-Pick diseases, gangliosidosis (including Tay-Sachs disease, and leukodystrophies; (B) Mucopolysaccharidoses, including Hunter syndrome and Hurler and Sanfilippo diseases; (C) Glycoprotein storage disorders; and (D) Mucolipidoses.

More specifically, example LSDs include sphingolipidoses, ceramidase, galactosialidosis, gangliosidoses, glucocerebroside, sphingomyelinase, sulfatidosis, mucopolysaccharidoses, mucolipidosis, lipidoses, oligosaccharide, lysosomal transport diseases, glycogen storage diseases, and cholesteryl ester storage disease.

In some embodiments, the LSD is mucopolysaccharidoses. Mucopolysaccharidoses (MPS) are caused by the absence or malfunctioning of lysosomal enzymes needed to break down glycosaminoglycans (GAGs). These long chains of sugar carbohydrates occur within the cells that help build bone, cartilage, tendons, corneas, skin and connective tissue. Seven distinct clinical types and numerous subtypes of the MPS have been identified. Examples include MPS I. MPS II, MPS III, MPS IV, MPS VT, MPS VII, and MPS TX.

MPS III, also known as Sanfilippo syndrome, is marked by severe neurological symptoms. These include progressive dementia, aggressive behavior, hyperactivity, seizures, some deafness and loss of vision, and an inability to sleep for more than a few hours at a time. This disorder tends to have three main stages. During the first stage, early mental and motor skill development may be somewhat delayed. Affected children show a marked decline in learning between ages 2 and 6, followed by eventual loss of language skills and loss of some or all hearing. Some children may never learn to speak. In the syndrome's second stage, aggressive behavior, hyperactivity, profound dementia, and irregular sleep may make children difficult to manage, particularly those who retain normal physical strength. In the syndrome's last stage, children become increasingly unsteady on their feet and most are unable to walk by age 10.

Thickened skin and mild changes in facial features, bone, and skeletal structures become noticeable with age. Growth in height usually stops by age 10. Other problems may include narrowing of the airway passage in the throat and enlargement of the tonsils and adenoids, making it difficult to eat or swallow. Recurring respiratory infections are common.

There are four distinct types of Sanfilippo syndrome, each caused by alteration of a different enzyme needed to completely break down the heparan sulfate sugar chain. Sanfilippo A is the most severe of the MPS III disorders and is caused by the missing or altered enzyme heparan N-sulfatase. Sanfilippo B is caused by the missing or deficient enzyme alpha-N-acetylglucosaminidase. Sanfilippo C results from the missing or altered enzyme acetyl-CoAlpha-glucosaminide acetyltransferase. Sanfilippo D is caused by the missing or deficient enzyme N-acetylglucosamine 6-sulfatase.

In some embodiments, the patient being treated has a neuropathophysiological condition such as dementia, aggressive behavior, hyperactivity, seizure, deafness or loss of vision.

In some embodiments, the patient may be identified as having decreased synaptic transmission or decreased levels of synaptic protein markers VGLUT1 (vesicular glutamate transporter 1), PSD-95 (postsynaptic density protein 95), VGAT (vesicular GABA transporter), SYN1 (Synapsin I), or Gephyrin. In some embodiments, the patient may be identified as having increased microgliosis, astrogliosis and ncuroinflammation. In some embodiments, the patient may be identified as having decreased activity or level of BDNF as compared to a reference healthy subject. In some embodiments, the treatment may be monitored by checking the activity level of BDNF in the patient, wherein increased BDNF indicates improvement of the disease.

In particular embodiments, the present disclosure provides a method for treating a mucopolysaccharidosis (MPS) III in a patient in need thereof, comprising intranasal administration to the patient an effective amount of a peptide that comprises the amino acid sequence of MEHFPGP (SEQ ID NO:1), or an analog thereof. Example analogs include MGHFPGP (SEQ ID NO:3), MEHFXPGP (SEQ ID NO:4). MGHFXPGP (SEQ ID NO:5), MEHFPAP (SEQ ID NO:6), MEHFXPAP (SEQ ID NO:7), and MGHFXPAP (SEQ ID NO:8), wherein X represents any amino acid residue. In some embodiments, the patient suffers from MPS IIIC.

In some embodiments, modified nootropic peptides are provided. For any of the peptides disclosed herein, it can be N-terminal acetylated and/or C-terminal amidated. Examples include N-terminal acetylated and/or C-terminal amidated MEHFPGP (SEQ ID NO:1), MGHFPGP (SEQ ID NO:3), MEHFXPGP (SEQ ID NO:4), MGHFXPGP (SEQ ID NO:5), MEHFPAP (SEQ ID NO:6), MEHFXPAP (SEQ ID NO:7), or MGHFXPAP (SEQ ID NO:8), wherein X represents any amino acid residue.

### Formulations

The present disclosure also provides pharmaceutical compositions suitable for intranasal administration. Upon intranasal administration, the agent may be retained in the submucous space of the nose, cross the arachnoid membrane, and enter into the central nervous system via the olfactory pathways. In some embodiments, a transport moiety complex is included to facilitate transport of the agent to the CNS, thereby improving response time and minimizing exposure of peripheral tissues to the active agents.

To increase the contact time and targeting to the olfactory nerves, formulation of a pharmaceutically active agent-transport moiety with a biocompatible adhesive or a delivery device can be prepared. The formulation may be in the form of a cream, liquid, spray, powder, or suppository which can be administered intranasally using a suitable applicator. Processes for preparing pharmaceuticals in these vehicles can be found throughout the literature. The formulation can be applied using any convenient method or device such as a spray device, metered dose applicator for cream, suppository suitable for intranasal insertion, and the like.

The formulation can also include a bioadhesive agent, for example, a mucoadhesive agent. The mucoadhesive agent permits a close and extended contact of the composition, or the drug released from said composition, with mucosal surface by promoting adherence of said composition or drug to the mucosa. The mucoadhesive agent is preferably a polymeric compound, such as preferably, a cellulose derivative but it may be also a natural gum, alginate, pectin, or such similar polymer. A preferred cellulose derivative is hydroxypropyl methylcellulose, commercially available from Dow Chemical Co. The mucoadhesive agent can be present in from about 5 to about 25%, by weight, preferably in from about 10 to about 15% and most preferably about 10%.

Bioadhesive microparticles or nanoparticles can constitute still another component of the intranasal formulations suitable for use in the present disclosure. The bioadhesive particles include derivatives of cellulose such as hydroxypropyl cellulose and polyacrylic acid and can provide sustained release of the pharmaceutically active agents for an extended period of time (possibly days) once they are placed in the appropriate formulation. A formulation comprising bioadhesive particles can provide a multi-phase liquid or semi-solid preparation which does not seep from the nose. The microparticles or nanoparticles cling to the nasal epithelium and can release the drug over extended period of time, for example, for several hours or more.

The biocompatible adhesives can include viscosity enhancers such as methylcellulose, sodium carboxymethylcellulose, chitosan, carbopol 934P and Pluronic 127. Thermogelling agents such as ethyl(hydroxyethyl) cellulose and Pluronic 127 can also be used to advantage. Thermogelling agents are liquid at room temperature and below, but at physiological temperatures (e.g., 32-37° C.), the viscosity of the solution increases such that the solution becomes a gel.

Pharmaceutical compositions may be formulated in combination with any suitable pharmaceutical vehicle, excipient or carrier that would commonly be used in this art, such as saline, dextrose, water, glycerol, ethanol, other therapeutic compounds, and combinations thereof. As one skilled in this art would recognize, the particular vehicle, excipient or carrier used will vary depending on the patient and the patient's condition, and a variety of modes of administration would be suitable for the compositions of the invention, as would be recognized by one of ordinary skill in this art.

Suitable nontoxic pharmaceutically acceptable excipients for use in the compositions of the present invention will be apparent to those skilled in the art of pharmaceutical formulations and examples are described in REMINGTON: The Science and Practice of Pharmacy, 20th Edition, A. R. Gennaro, ed., (2000).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1. Preliminary Study in Mouse MPSIIIC Models

This example developed and studied a mouse model of Mucopolysaccharidosis IIIC (MPSIIIC or Sanfilippo disease C) specifically a “knockout” strain Hgsnat-Geo and a “knock-in” strain Hgsnat^{P304L} (Hgsnat^{P311L}) expressing mouse HGSNAT enzyme with an analog of human missense mutation Pro311Leu. The result demonstrated that the pathophysiological mechanism of the disease involves both neurodegeneration and functional pathological changes in the CNS affecting synaptogenesis, synaptic transmission, neuroinflammation, learning and memory deficits. Mice also have some pathologies of peripheral tissues including splenomegaly. It also demonstrated that these pathologies were reversed by treating MPSIIIC mice with a peptide AVP6 (an acetylated Semax, a nootropic peptide having the amino acid sequence of MEHFPGP (SEQ ID NO:1); the N-terminal M is acetylated which is shown to increase the activity and stability of the peptide in preliminary studies).

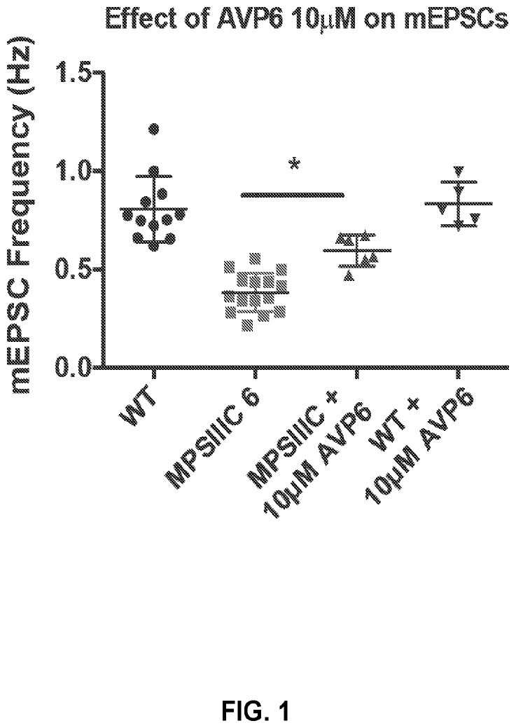

Restoration of synaptic deficits: MPSIIIC mice in comparison with wild-type (WT) control mice demonstrate deficits in synaptic transmission in the CA1 neurons of the hippocampus, a brain region implicated in the formation and storage of memory. As shown in FIG. 1, however, acute bath application of AVP6 on hippocampal slices from MPSIIIC mouse caused a significant restoration of reduced amplitude and frequency of miniature excitatory postsynaptic currents (mEPSC).

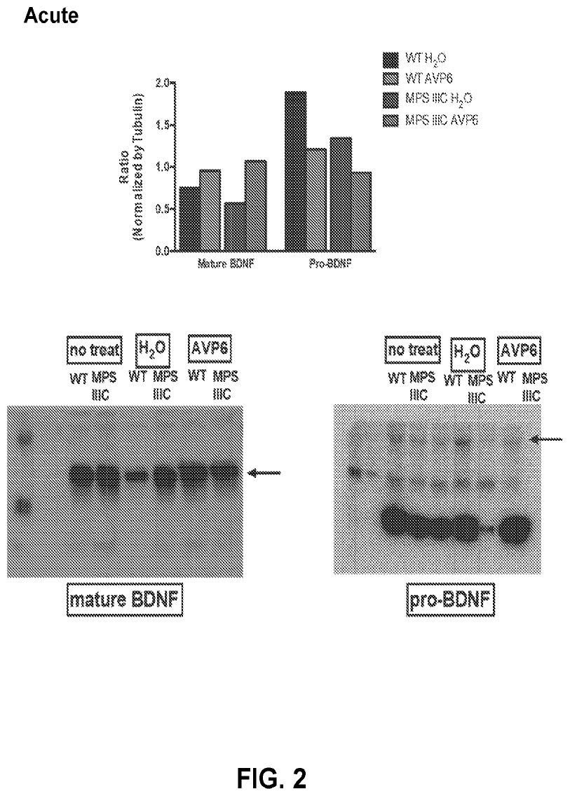

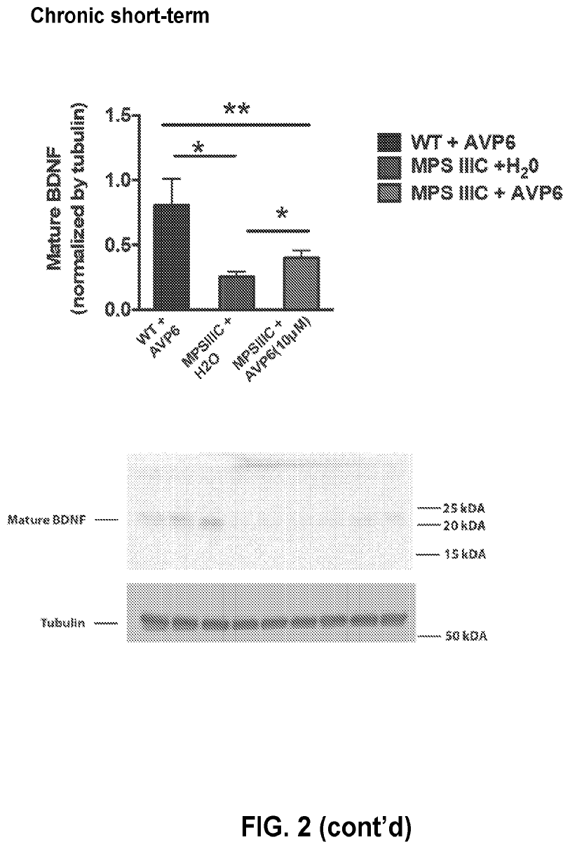

Restoration of deficits in neurotrophic molecules: Neurotrophic molecules such as BDNF (Brain Derived Neurotrophic Factor) are important for neuronal development and neuroplasticity mechanisms of memory. In MPSIIC mice, it was observed that the levels of the pro form of BDNF (proBDNF) were increased while the levels of the mature BNDF were decreased. In contrast, upon acute (24 h) or chronic short-term (10 days) intranasal administration of AVP6, mature BDNF levels in MPSIIIC mice were found to be significantly increased (FIG. 2).

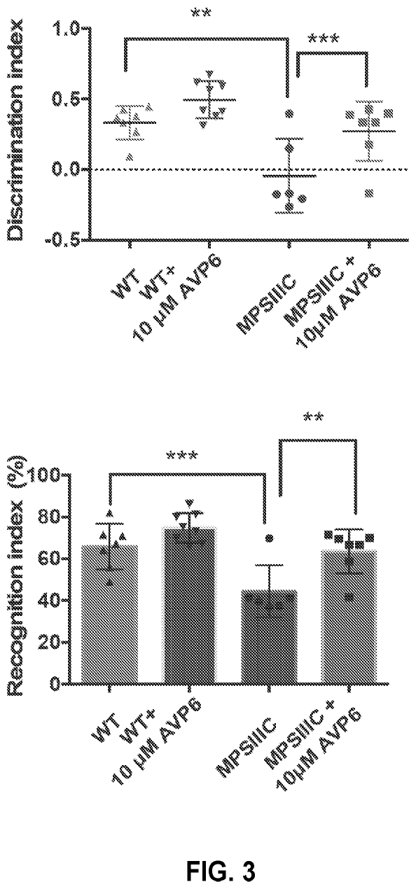

Restoration of neurobehavioral deficits: MPSIIIC patients suffer from progressive cognitive decline and other neurobehavioural deficits eventually leading to dementia. Similarly, the MPSIIIC mice exhibited a significantly reduced discrimination index in the Novel Object Recognition task, a behavioral test that evaluates working memory in rodent models of neurological disorders. The task relies on the natural inclination of rodents to explore a novel object than a familiar one. Therefore, failure to discriminate the old object from the new one reflects a reduced learning or recognition memory. Importantly, intranasal administration of AVP6 for 10 days significantly recovered memory deficits in these animals (FIG. 3).

### Example 2. Preclinical Studies

This example shows the results of preclinical studies of evaluating the neurorestorative properties of AVP6 in mouse models of MPS IIIC Hgsnat-Geo and Hgsnat^{P304L}, which closely mimic the pathological course of the disease in humans.

I. Effect of AVP6 on Synaptic Neurotransmission

### A. Miniature Synaptic Events

Models and developmental time points: Hippocampal slices from Hgsnat-Geo and Hgsnat^{P304L} mice; P14-20 and P45-60.

Method: Whole cell voltage clamp electrophysiology recordings (miniature synaptic currents) were conducted on CA1 pyramidal neurons in acute hippocampal slices.

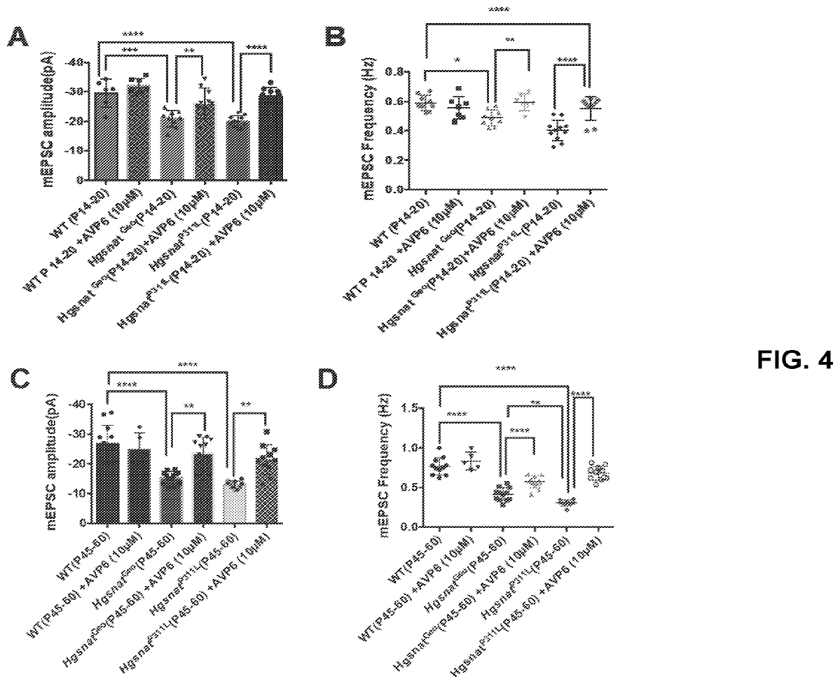

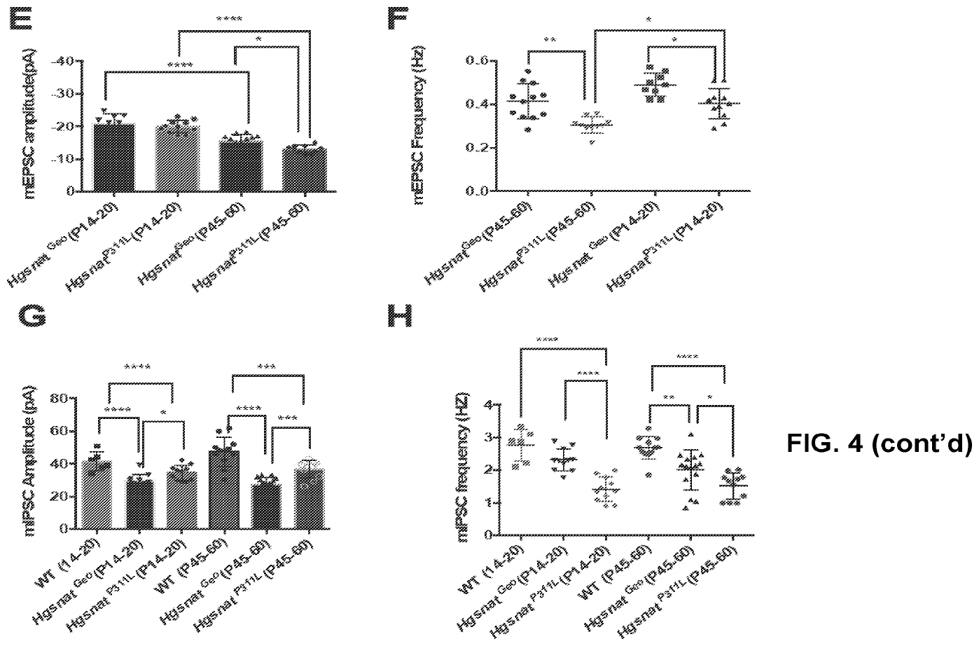

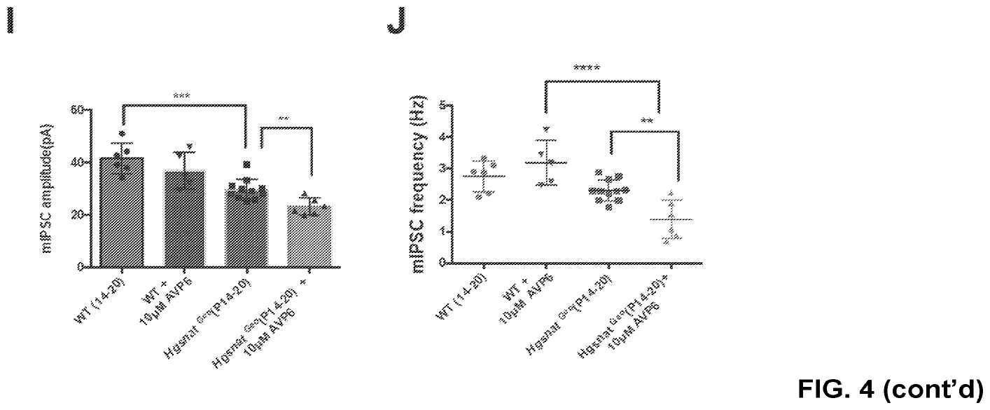

Results:
i. At P14-20, miniature excitatory post synaptic current (mEPSC) frequency and amplitude was reduced in Hgsnat^{Geo} mice compared to WT animals. Hgsnat^{P311L} mice also displayed reduced mEPSC amplitude and frequency as compared to WT controls (FIG. 4A and B).
ii. At P14-20, AVP6 bath application on slices at 10 μM final concentration recovered deficits in mEPSC amplitude and frequency in both Hgsnat^{P311L} and Hgsnat^{Geo} mice (FIG. 4A and B).
iii. At P45-60, mEPSC frequency and amplitude were reduced in Hgsnat^{Geo} and Hgsnat^{P311L} mice as compared to WT. Additionally, at P45-60, Hgsnat^{P311L} mice revealed, significantly reduced mEPSC amplitude and frequency compared to age-matched Hgsnat^{Geo} mice. Hgsnat^{P311L} mice also revealed significantly reduced mEPSC frequency and amplitude at P45-60 as compared with P14-20. (FIG. 4C, D, E, F).
iv. At P45-60, AVP6 bath application on slices at 10 μM recovered deficits in mEPSC amplitude and frequency in both Hgsnat^{Geo} and Hgsnat^{P311L} mice. A trend for an increase in mEPSC amplitude and frequency was noticed in WT animals. (FIG. 4C and D).
v. At P14-20, miniature inhibitory postsynaptic current (mIPSC) amplitude and frequency was found to be significantly reduced in Hgsnat^{P311L} mice as compared to Hgsnat^{Geo} and WT controls. At P 45-60, both mIPSC amplitude and frequency was also significantly reduced in Hgsnat^{P311L} mice as compared to both WT controls and Hgsnat^{Geo} mice (FIG. 4G and H).
vi. AVP6 bath application on slices at 10 μM did not recover deficits in mIPSC amplitude and frequency at P14-20 in Hgsnat^{Geo} mice and therefore the drug has not been tested for other age groups or for the Hgsnat^{P311L} model.

This example demonstrates that excitatory (glutamatergic) synaptic neurotransmission and inhibitory neurotransmission was impaired in Hgsnat^{Geo} and Hgsnat^{P311L} mice at both P14-20 and P45-60. AVP6 rescued deficits in glutamatergic neurotransmission at both ages in both the animal models.

### B. Evoked Synaptic Events

Model and Developmental time points: Hippocampal slices from Hgsnat^{Geo} and Hgsnat^{P311L} mice; P14-20.

Method: Synaptic currents (composite glutamatergic EPSCs, AMPA EPSCs and NMDA EPSCs) were evoked by stimulating Schaffer collaterals and recordings were conducted from the hippocampus CA1 pyramidal cells in hippocampal slices from Hgsnat^{Geo} and Hgsnat^{P311L} mice. Paired pulse stimulation protocol with increasing stimulus intervals was used to identify the locus of deficit.

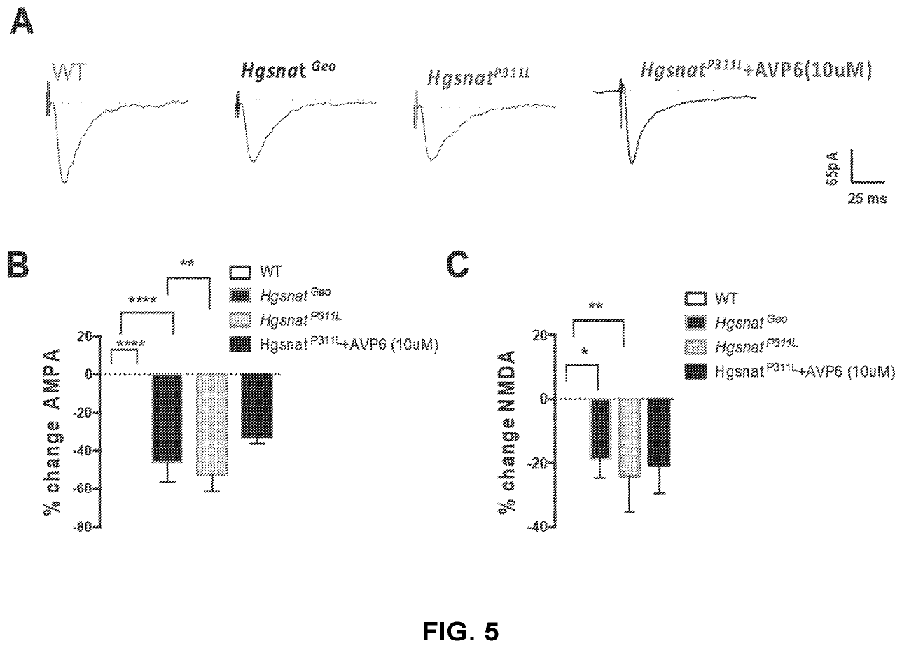

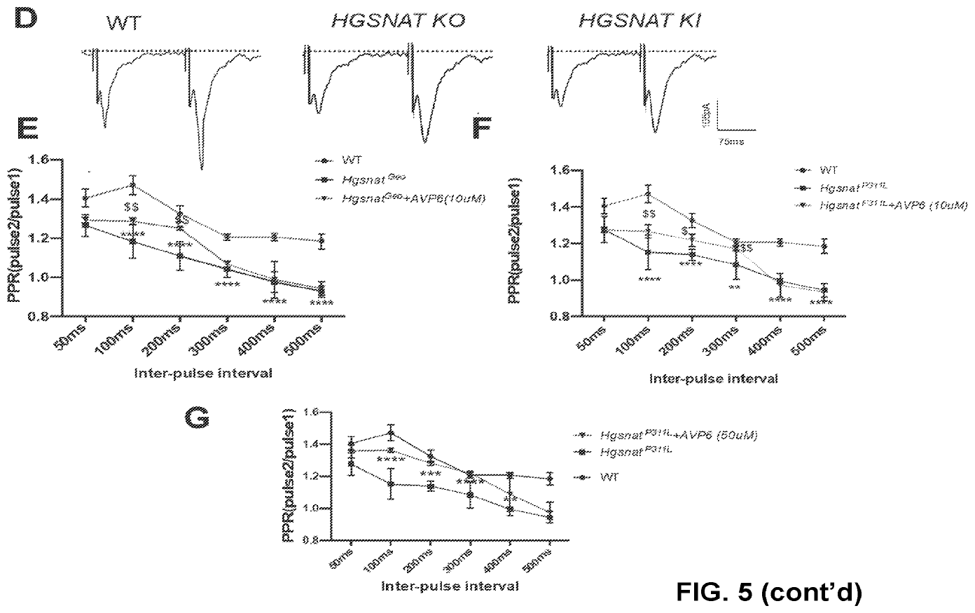

Results:
i. At P14-20, evoked AMPA and NMDA currents were found to elicit significantly reduced amplitudes in both Hgsnat^{Geo} and Hgsnat^{P311L} mice as compared to WT animals upon the same intensity of stimulation (FIG. 5B and C).
ii. Bath application of AVP6 at 10 μM concentration recovered deficits AMPA but not in NMDA currents in Hgsnat^{P311L} mice (FIG. 5B and C).
iii. Upon administering paired pulse stimulation protocols, synaptic facilitation was observed in WT, Hgsnat^{Geo} and Hgsnat^{P311L} mice. However, paired pulse ratios were significantly reduced in Hgsnat^{Geo} and Hgsnat^{P311L} mice as compared to WT controls (FIG. 5D).
iv. Ten μM AVP6 significantly recovered PPF deficits in at lower (100-200 ms in Hgsnat^{Geo} mice and 100-300 ms in Hgsnat^{P311L} mice) inter-pulse interval (IPI) but not at higher (400 and 500 ms) IPI (FIG. 5E and F; * indicates significance with comparison to WT; $ indicates significance for AVP6 treatment).
v. When concentration of AVP6 was increased to 50 μM, PPF deficits were rescued at 100, 200, 300 and 400 ms but not at 500 ms IPI (FIG. 5G; * indicates significance of AVP6 treatment).
vi. Upon administering AVP6 at 50 μM, the recording was lost in 5 of 11 cells suggesting certain level of neurotoxicity of AVP6 at 50 μM concentration.

This example shows that both AMPA and NMDA currents were reduced in Hgsnat^{P311L} and Hgsnat^{Geo} mice but AMPA deficits were predominant. Changes in paired pulse ratio suggest that the deficits have presynaptic origin.

AVP6 at 10 μM rescued AMPA current deficits but not NMDA current deficits in Hgsnat^{P311L} mice. AVP6 at 10 μM rescued presynaptic deficits at lower IPI but not at higher IPI. Increased 50 μM doses of AVP6 rescued presynaptic deficits at longer IPI range but exerted some cytotoxicity.

Together, these data show that AVP6 rescues excitatory synaptic transmission by acting at presynaptic AMPA receptors.

2. Effect of AVP on Synaptic Morphology and Synaptic Proteins In Vitro:

Model and Developmental time points: Primary hippocampal neuronal cultures established from E16 embryos of Hgsnat^{P311L} mice.

Method: The hippocampal neurons were cultured until DIV (Day In Vitro) 21 and 50% of media was changed every 3 days. AVP6 at a final concentration of 10 μM was added to the media when plating and during every media change. At DIV21 neurons were fixed and analysed by immunohistochemistry using markers of dendrites (MAP), axons (neurofilament protein, NF-H), and synaptic transmission (PSD95 and BDNF). Synaptic spine architecture and additional protein synaptic markers (Vglut/PSD95 and VAMP) will be studied.

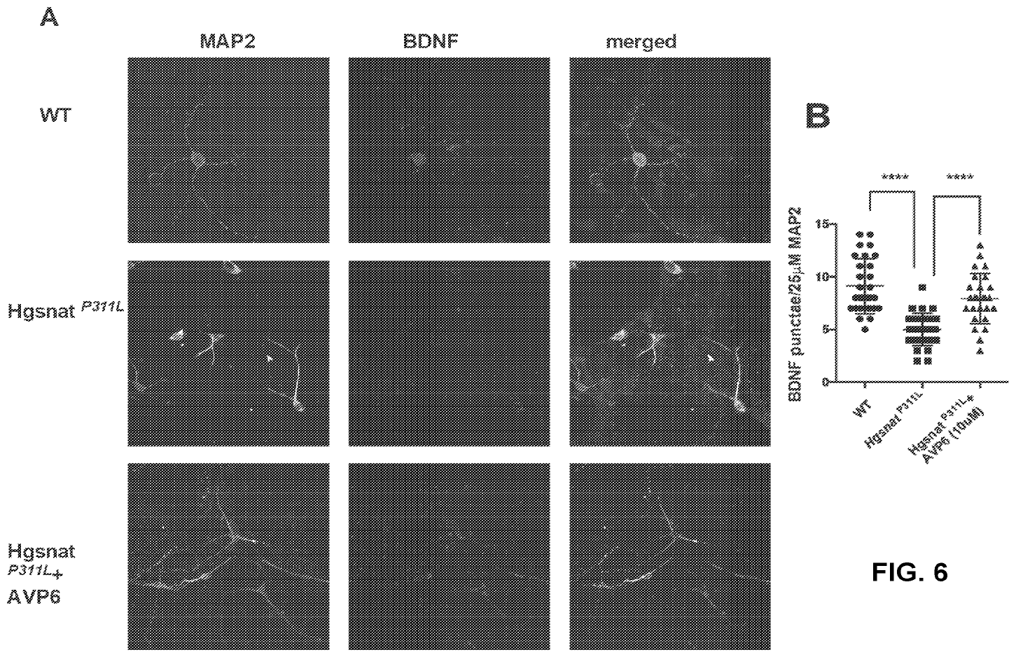

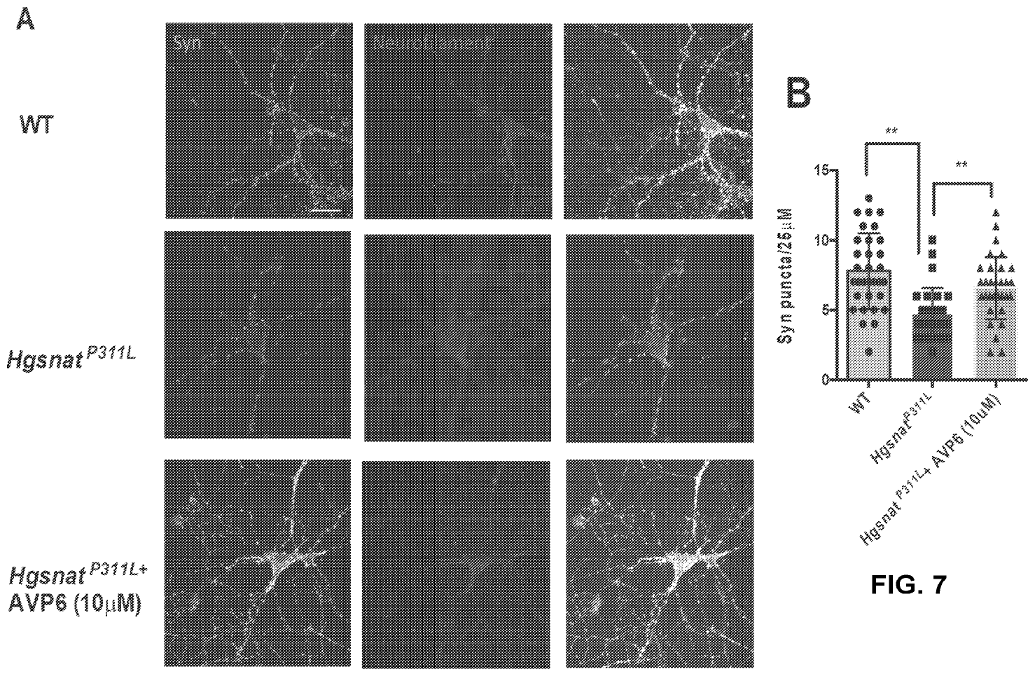

Results:
i. In Hgsnat^{P311L} neuronal cultures, the number of BDNF-positive punctae was reduced as compared to WT neurons (FIG. 6A and B).
ii. AVP6 treated Hgsnat^{P311L} neurons showed increase in the number of BDNF-positive punctae (FIG. 6A and B).
iii. In Hgsnat^{P311L} neuronal cultures, the number of Synapsin 1-positive punctae was reduced as compared to WT neurons (FIG. 7A and B).
iv. AVP6 treated Hgsnat^{P311L} neuronal cultures showed increase in Synapsin 1-positive puncta (FIG. 7A and B).

This example shows that primary neuronal cultures from Hgsna^{P311L} mice showed drastic reduction of BDNF, a neurotrophic factor expressed in hippocampus, cortex and other areas implicated in learning and memory and implicated in neuronal survival, growth and differentiation, as well as in formation of new synapses during memory processes. Treatment with 10 μM AVP6 rescued the BDNF deficit.

Synapsin 1, a neuronal phosphoprotein that coats synaptic vesicles and is known to modulate neurotransmitter release, was reduced in cultured Hgsnat^{P311L} neurons. AVP6 treatment rescued Synapsin 1 deficit as well, consistent with reported above AVP-mediated induction of miniature and evoked excitatory currents at the presynaptic side.

3. Behavioural Effects of Acute and Short-Term AVP6 Administration In Vivo.

### Acute Behavioural Studies

Hyperactivity and reduced anxiety. Model and Developmental time points: Hgsnat^{P311L} mice; 4-month and 6-month-old.

Method: Open field test that explores behaviour of animals in open area. Rodents normally avoid the center of the arena (anxiety reflex) and spend certain time immobile (freezing reflex). AVP6 solution in saline was administered intranasally at 50 μg/kg (5 μl/nostril) to the animals 17 h before the experiment.

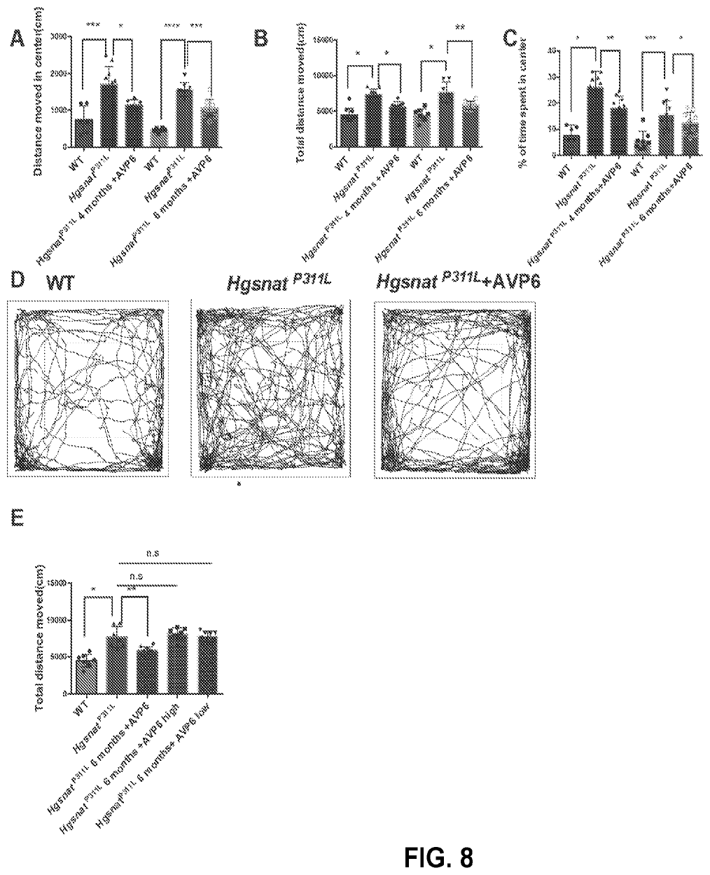

Results:
i. Hgsnat^{P311L} mice at 4 months and 6 months show significantly increased hyperactivity (increase in total distance traveled) and reduced anxiety (increased time spent in the center of the arena and increased distance traveled in the center of the arena) as compared to WT animals (FIG. 8A, B and C).
ii. AVP6 treatment rescues hyperactivity and reduced anxiety in Hgsnat^{P311L} mice at both developmental time points. (FIG. 8A, B, C). Panel D shows representative track images of mouse movement in the open field arena for 4-months-old WT, Hgsnat^{P311L} and AVP6-treated Hgsnat^{P311L} mice.
iii. Increased (500 μg/kg) or reduced (10 μg/kg) doses of AVP6 fail to rescue hyperactivity or reduced anxiety in 6-months-old Hgsnat^{P311L} mice (FIG. 8E).

This example shows that single intranasal administration of AVP6 in a dose of 50 μg/kg 17 h before the experiment rescued hyperactivity and reduced anxiety in Hgsnat^{P311L} mice at the age of 4 months and 6 months.

### Reduced Fear

Model and Developmental time points: Hgsnat^{Geo} (4 months, 6 months and 8 months), Hgsnat^{P311L} mice (4 months and 6 months).

Method: Elevated plus maze that measures a natural fear of heights reflex of animals. AVP6 was administered intranasally at 50 μg/kg (5 μl/nostril) to the animals 17 h before the experiment.

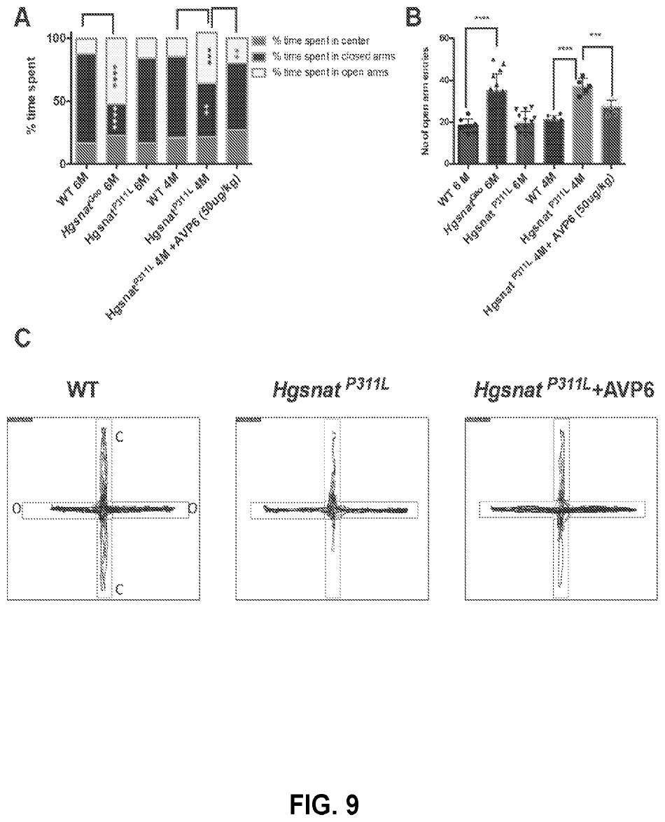

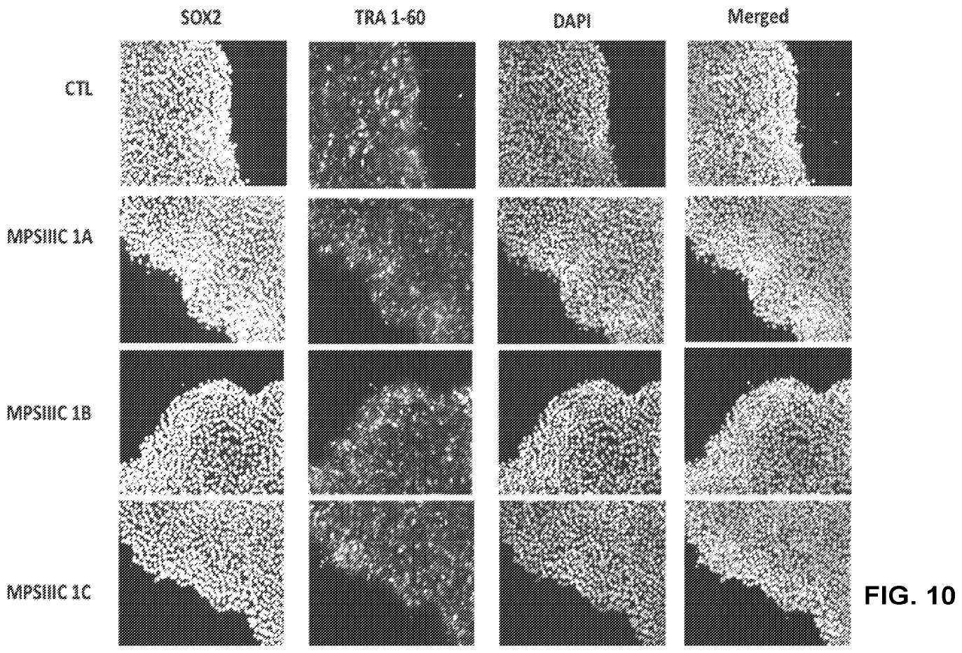

Results:
i. Hgsnat^{P311L} mice at 4 months show significantly reduced fear (increase in the time spent in open arms and increase in the number of open arm entries) as compared to WT animals (FIG. 9A and B; * indicates comparison to WT, indicates comparison to Hgsnat^{P311L}).
ii. Hgsnat^{Geo} mice reveal reduced fear at 6 months but age matched Hgsnat^{P311L} mice do not (FIG. 9A and B).
iii. AVP6 treatment rescues reduced fear in Hgsnat^{P311L} mice at 4 months (FIG. 9A, B; Panel C shows representative track images of movement in the elevated plus maze for WT, Hgsnat^{P311L} and AVP6-treated Hgsnat^{P311L} 4-month-old mice).

The phenotype of reduced fear in Hgsnat^{Geo} mice was present at 6 months. In Hgsnat^{P311L} mice it was present at 4 months and is lost at 6 months, suggesting a more rapidly progressing and severe phenotype for this model as compared to Hgsnat^{Geo} mice.

Single intranasal administration of AVP6 in a dose of 50 μg/kg rescued reduced fear at 4 months in Hgsnat^{P311L} mice.

4. Behavioural Effects of Short-Term AVP6 Administration In Vivo.

### Working Memory

Model and Developmental time points: Hgsnat^{Geo} mice; 4-month-old.

Method: Novel object recognition test that studies working memory of mice by measuring their ability to discriminate a familiar from a novel object. AVP6 was administered intranasally at a daily dose 50 μg/kg (5 μl/nostril) for 10 consecutive days before the experimental day.

Results:
i. Hgsnat^{Geo} mice at 4 months show significantly reduced discrimination and recognition indexes as compared to WT animals (FIG. 3).
ii. Ten-day treatment with AVP6 rescues working memory deficits in 4-months-old Hgsnat^{Geo} mice (FIG. 3).

This example shows that short term treatment regimen with AVP6 (10 days at 50 μg/kg) rescued working memory deficit in Hgsnat^{Geo} mice at 4 months.

5. Effect of AVP6 on BDNF Regulation In Vivo:

### Model and Developmental Time Points: Hgsnat^{Geo} (4 Months)

Method: AVP6 was administered intranasally at 50 μg/kg (5 μl/nostril) for 10 consecutive days. After sacrifice, changes in the levels of BDNF, a protein involved in long-term synaptic potentiation and memory consolidation, in the dissected hippocampi of mice were analyzed by Western blots.

Results: Hgsnat^{Geo} mice at 4 months show reduced levels of mature BDNF in hippocampus as compared to WT animals. 10-day intranasal treatment with AVP6 at a daily dose of 50 μs/kg increases BDNF levels (FIG. 2).

AVP6 at 50 μg/kg partially restored reduced BDNF levels in Hgsnat^{Geo} mice at 4 months consistent with the ability of the drug to rescue deficit in a working memory. This example also shows that BDNF can be used as one of predictive biomarkers for AVP6 efficacy studies.

6. Generation and Characterization of iPSCs from Skin Fibroblasts of MPS MC Patients (3 Lines) and Healthy Controls (2 Lines):

This example attempted to carry out immunohistochemical characterization for pluripotency markers (TRA1-60. SOX2), HGSNAT activity and mutations, karyotyping, and immunohistochemical characterization of iPSC in vitro differentiation to the three germ layers (ectoderm, mesoderm, endoderm).

Immunohistochemical analysis confirmed all cell lines are positive for pluripotency markers TRA1-60 and SOX2 (FIG. 10).

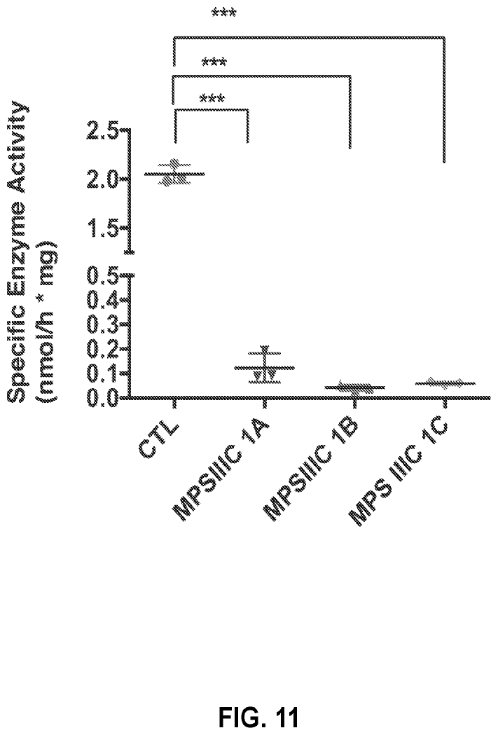

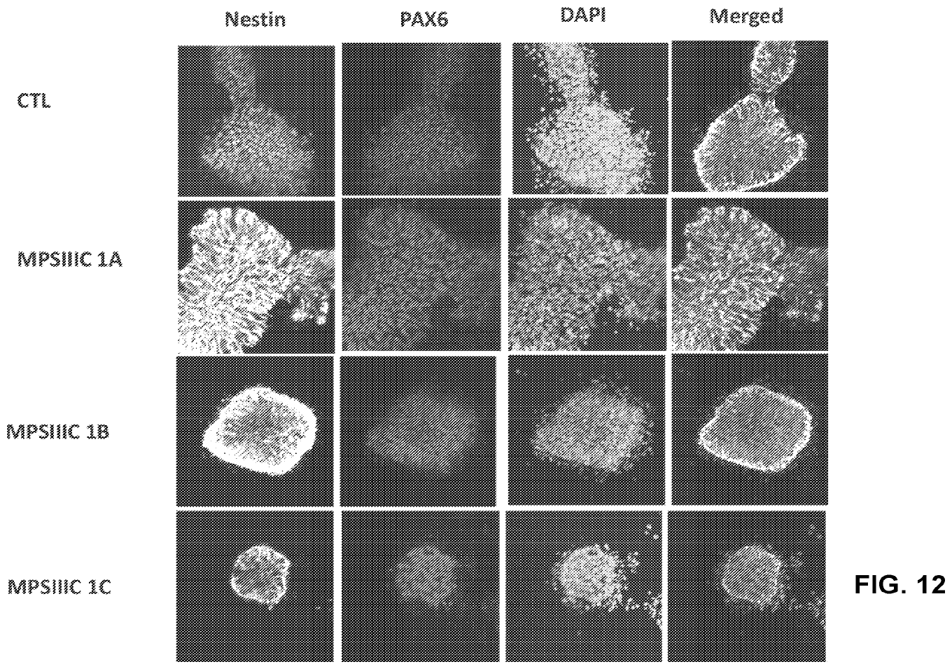

Specific HGSNAT activity in iPSC derived from skin fibroblasts of three MPSIIIC patients was significantly reduced as compared with healthy control (FIG. 11).

The following deleterious variants in the HGSNAT gene have been identified in the MPSIIIC patients:
i. MPS IIIC 1A: compound heterozygous for c.234+5G>A (exon 2-intron 2 boundary) and c.1411G>A; p.E471K
ii. MPSIIIC 1B: compound heterozygous for c.118+1G>A (g.43140615 G>A) (intron 1) and c.1622C>T (g. 43197848 C>T); p.S541L in exon 17
iii. MPS IIIC 1C: homozygous for c234+1G>A present in cis with benign c.710C>A/g. 43170661 C>A variant in Exon 7 resulting in p.P237Q change.

Immunohistochemical Characterization of iPSC Differentiation In Vitro to the Three Germ Layers (Ectoderm, Mesoderm, Endoderm).

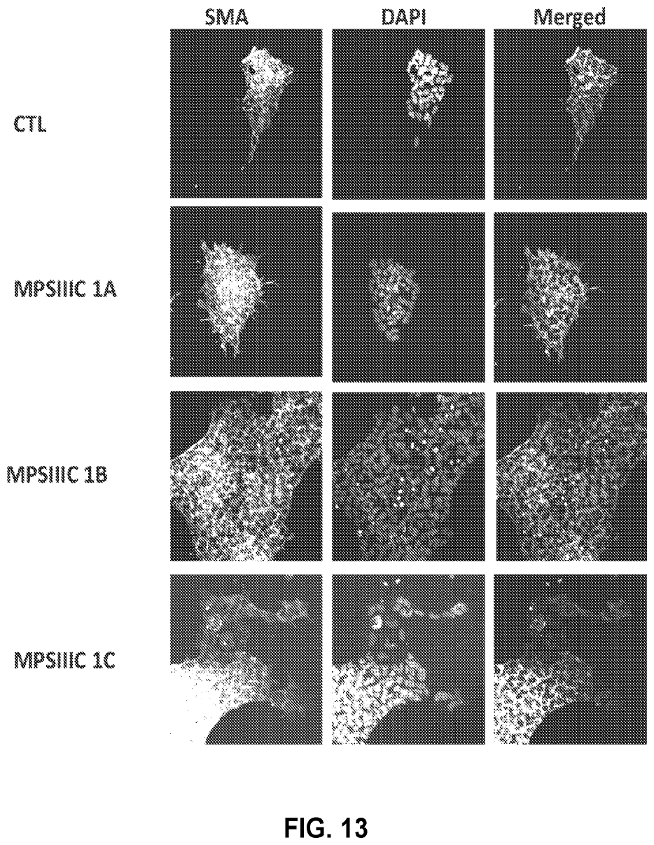

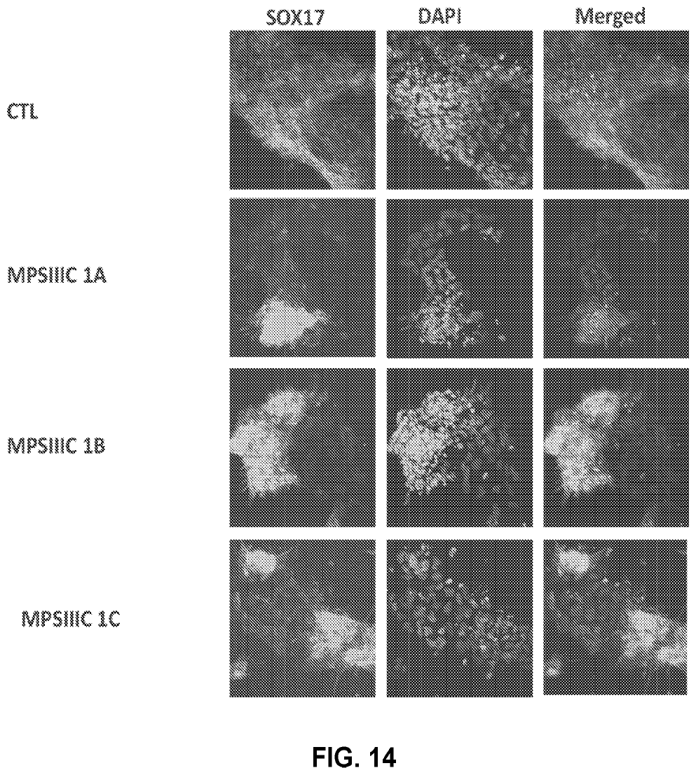

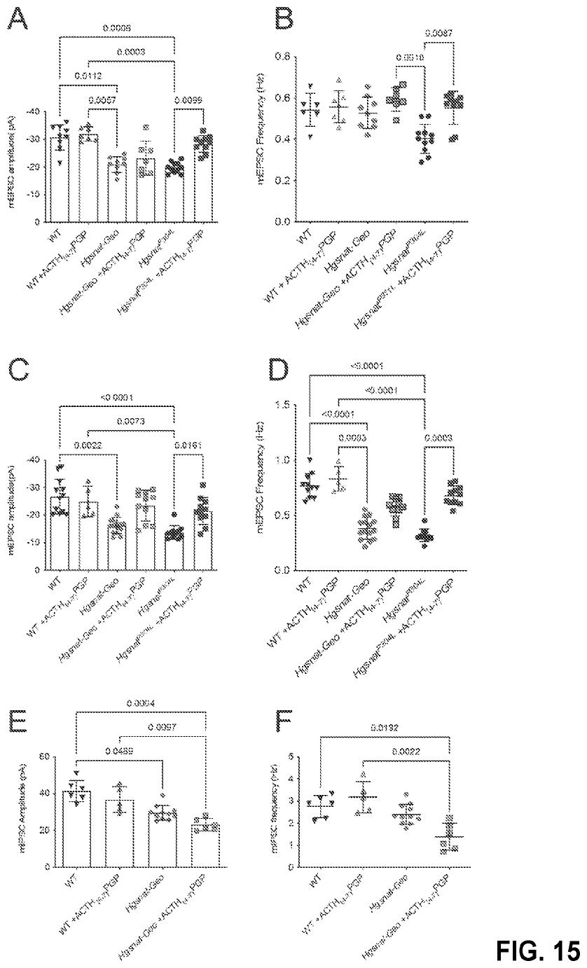

In-vitro differentiation of iPSCs into ectoderm cells positive for Nestin and Pax6 protein markers is shown in FIG. 12. In-vitro differentiation of iPSCs into mesoderm cells was positive for SMA (smooth muscle actin) protein marker is shown in FIG. 13. In-vitro differentiation of iPSCs into endoderm cells was positive for SOX17(CXCR4) protein marker is shown in FIG. 14.

In conclusion, at the synaptic level, the above data show that bath application of AVP6 rescues deficits in glutamatergic neurotransmission in acute hippocampal slices of both Hgsnat^{Geo} and Hgsnat^{P311L} mice. AVP6 acts on excitatory neurotransmission processes and does not ameliorate deficits in inhibitory neurotransmission processes. AVP6 preferentially rescues deficits in AMPA currents, likely through presynaptic mechanisms by increasing synaptic vesicle release from the readily releasable pool. AVP6 rescues reduced levels of BDNF in hippocampal neuronal cultures from Hgsnat^{P311L} mice. AVP6 also rescues reduced levels of Synapsin 1 in neuronal cultures from Hgsnat^{P311L} mice, consistent with the hypothesis that the drug restores levels of proteins involved in neurotransmitter release.

At the behavioural level, single acute 50 μg/kg dose of AVP6 (but not lower or higher doses of 10 μg/kg or 500 μg/kg) rescues anxiety and fear deficit and hyperactivity at both 4 and 6 months in Hgsnat^{P311L} mice. 10-day treatment with AVP6 at 50 μg/kg rescues impairment of working memory in Hgsnat^{Geo} mice.

At the molecular level, ten-day treatment with AVP6 at 50 μg/kg increases levels of BDNF in Hgsnat^{Geo} mice. BDNF can be used as a predictive biomarker in AVP6 efficacy studies.

Further, iPSCs have been successfully engineered from skin fibroblasts of 3 MPSIIIC patients and one healthy control. Pluripotency of iPSCs have been confirmed by their ability to differentiate in vitro into three major germ layers. HGSNAT enzyme activity levels in the MPSIIIC iPSC lines are significantly reduced as compared with the control line.

### Example 3. AVP6 Delays Neurological Manifestations in MPS III by Rescuing Glutamatergic Neurotransmission Defects, Increasing Synaptogenesis, Reducing Neuroinflammation and Preventing Neuronal Death

The instant inventors developed a MPS IIIC murine model, Hgsnat^{Geo} (a functional knockout of the Hgsnat locus in C57Bl/6N mice), which characterized the pathophysiology of the disease of MPS III in human patients. The results show that accumulation of HS in microglia in MPS IIIC brain triggers a cascade of downstream pathogenic reactions in neurons leading, eventually, to their death.

The previous examples characterized a new and more severe knock-in mouse model of MPS IIIC matching more aggressive early-onset clinical phenotype of MPS IIIC patients. These mice (Hgsnat^{P304L}) are homozygous for an analog of pathogenic human mutation Pro311Leu. Compared to the Hgsnat^{Geo} mice, Hgsnat^{P304L} mice of similar age have increased HS levels, lysosomal storage and neuroinflammation. Hgsnat^{P304L} mice also have an earlier onset of memory impairment and hyperactivity, and their survival is reduced by about ˜20-weeks.

The data demonstrate drastically reduced synaptic activity in the pyramidal CA1 hippocampal neurons in both Hgsnat^{Geo} and Hgsnat^{P304L} mouse models of MPS IIIC. The defects are observed for both excitatory (mEPSCs) and inhibitory (mIPSCs) miniature synaptic currents already at P (postnatal day) 45-60, 2-3 months before the development of other neuronal pathologies. These data are supported by the marked reduction in the VGLUT1/PSD-95 puncta in hippocampal neurons of MPS IIIC mice, together, suggesting overall synaptic deficits that aggravate with age. Moreover, density of dendritic synaptic spines (which typically receive input from excitatory synapses) of pyramidal CA1 hippocampal neurons is reduced already at P10 and never reaches levels observed in WT mice. Drastically reduced levels of synaptic vesicles in the terminals and smaller areas of postsynaptic densities were also found in pyramidal CA1 hippocampal neurons at 3 and 6 months. These changes affect mainly excitatory circuits.

PSD-95 (the protein which is highly enriched at excitatory postsynaptic sites) is the most severely reduced biomarker, not only in the mouse model but also in all studied post-mortem cortical tissues of neurological MPS patients. Other reduced biomarkers are synaptic vesicle protein Syn1, VGLUT1, and BDNF, a neurotrophic factor expressed in hippocampus, cortex and other areas implicated in learning and memory. BDNF promotes neuronal survival, growth and differentiation, as well as formation of new synapses during memory processes. Together, these experiments demonstrate that lysosomal storage in CA1 hippocampal pyramidal neurons of MPS IIIC mice results in appearance of early and drastic synaptic defects.

This example demonstrates that in brain slices from MPS IIIC mouse models AVP6 reduced amplitude and frequency of miniature excitatory postsynaptic currents and evoked excitatory postsynaptic currents in pyramidal CA1 neurons. AVP6 also reversed the decrease in synaptic protein levels in cultured MPS IIIC mouse hippocampal neurons and in iPSC-derived cortical neurons of human MPS III A and MPS IIIC patients. Furthermore, daily intranasal administration of this peptide reduced hyperactivity and rescued defects in working and spatial memory in MPS IIIC mice at 4 months and 6-7 months and delays progression of brain pathology.

### Materials and Methods

### Murine Models

Approval for animal experimentation was granted by the Animal Care and Use Committee of the Ste-Justine Hospital Research Center. Mice were housed in an enriched environment with continuous access to food and water, under constant temperature and humidity, on a 12 h light/dark cycle. Mice were kept on a normal chow diet (5% fat, 57% carbohydrate). Hgsnat^{P304L} knock-in C57Bl/6J mouse strain generated at McGill Integrated Core for Animal Modeling (MICAM) used CRISPR/Cas9 technology, targeting exon 9 of the Mus musculus heparan sulfate acetyl-CoA: alpha-glucosaminide N-acetyltransferase (Hgsnat) gene.

### Enzyme Activity Assays

The specific enzymatic activities of HGSNAT, β-hexosaminidase, and β-galactosidase were assayed essentially as follows. Tissues extracted from mice or pellets of cultured cells were snap-frozen in liquid nitrogen before storage at −80° C. Fifty mg samples were homogenized in 250 μl of H₂O using a sonic homogenizer (Artek Systems Corporation). For HGSNAT assays, 5 μl aliquots of the homogenates were combined with 5 μl of McIlvain Buffer (pH 5.5), 5 μl of 3 mM 4-methylumbelliferyl-β-D-glucosaminide (Moscerdam), 5 μl of 5 mM acetyl-coenzyme A and 5 μl of H₂O. The reaction was incubated for 3 h at 37° C. stopped with 975 μl of 0.4 M glycine buffer (pH 10.4), and fluorescence was measured using a ClarioStar plate reader (BMG Labtech). Blank samples were incubated without the homogenates which were added after the glycine buffer.

The activity of β-hexosaminidase was measured by combining 2.5 μl of 10× diluted homogenate (˜2.5 ng of protein) with 15 μl of 0.1 M sodium acetate buffer (pH 4.2), and 12.5 μl of 3 mM 4-methylumbelliferyl N-acetyl-β-D-glucosaminide (Sigma-Aldrich) followed by incubation for 30 min at 37° C. The reaction was stopped 0.4 M glycine buffer (pH 10.4) and fluorescence was measured as above.

The activity of acidic β-galactosidase was measured by adding 12.5 μl of 0.4 M sodium acetate, 0.2 M NaCl (pH 4.2) and 12.5 μl of 1.5 mM 4-methylumbelliferyl β-D-galactoside (Sigma-Aldrich) to 10 μl of 10× diluted homogenate (˜1 ng of protein). After 15-min incubation at 37° C., the reaction was stopped with 0.4 M glycine buffer (pH 10.4) and fluorescence was measured as above.

### Behavioral Analysis

The spontaneous alternation behavior, spatial working memory and exploratory activity of mice were evaluated using a white Y-maze as follows. The maze consisted of three identical white Plexiglas arms (40×10×20 cm, 120° apart) under dim lighting conditions. Each mouse was placed at the end of one arm, facing the center, and allowed to explore the maze for 8 min. All experiments were performed at the same time of the day and by the same investigator to avoid circadian and handling bias. Sessions were video-recorded and arm entries were scored by a trained observer, unaware of the mouse genotype or treatment. Successful alternation was defined as consecutive entries into a new arm before returning to the two previously visited arms.

Alternation was calculated as: [number of alternations/total number of arm entries−2]×100.

Novel object recognition test was used for assessing short-term recognition memory. Mice were placed individually in a 44×33×20 cm (length×width×height) testing chamber with white Plexiglas walls for 10 min habituation period and returned to their home cage. The next day, mice were placed in the testing chamber for 10 min with two identical objects (red plastic towers, 3×1.5×4.5 cm), returned to the home cages, and 1 hour later, placed back into the testing chamber in the presence of one of the original objects and one novel object (a blue plastic base, 4.5×4.5×2 cm) for 10 min. After each mouse, the test arena as well as the plastic objects were cleaned with 70% ethanol to avoid olfactory cue bias. The discrimination index (DI) was calculated as the difference of the exploration time between the novel and old object divided by total exploration time. A preference for the novel object was defined as a DI significantly higher than 0. Mice who showed a side preference, noted as a DI of ±0.20 during familiarization period, and those who had a total exploration times lower than 3 seconds were excluded from analysis.

The open-field test was performed as previously described. Briefly, mice were habituated in the experimental room for 45 mins before the commencement of the test. Each mouse was then gently placed in the center of the open-field arena and allowed to explore for 20 min. The mouse was removed and transferred to its home cage after the test, and the arena was cleaned with 70% ethanol before the commencement of the next test. Analysis of the behavioral activity was done using the Smart video tracking software (v3.0, Panlab Harvard Apparatus), and total distance traveled and percent of time spent in the center zone were measured for hyperactivity and anxiety assessment, respectively.

The elevated plus-maze test was performed as follows. Each mouse was placed in the center of the elevated plus maze and allowed to freely explore undisturbed for 10 min. After each testing, the mouse was returned to the home cage and the arena was cleaned with 70% ethanol before the commencement of the next test. Analysis of the behavioral activity (percentage of time spent in the center zone, closed arms, and open arms; as well as the number of open arm entries) was done by the Smart v3.0 software.

### Transmission Electron Microscopy

At 3 and 6 months, three mice from each group were anesthetized with sodium pentobarbital (50 mg/kg BW) and perfused with PBS, followed by 2.5% glutaraldehyde in 0.2 M phosphate buffer (pH 7.2). The brains were extracted and post-fixed in the same fixative for 24 h at 4° C. The hippocampi were dissected, mounted on glass slides, stained with toluidine blue and examined on a Leica DMS light microscope to select the CA1 region of the hippocampus for electron microscopy. The blocks were further embedded in Epon, and 100 nm ultrathin sections were cut with an Ultracut E ultramicrotome, mounted on 200-mesh copper grids, stained with uranyl acetate (Electron Microscopy Sciences) and lead citrate, and examined on a FEI Tccnai 12 transmission electron microscope. For quantification, the micrographs were taken with 13,000× and 30,000× magnification.

### Mouse Primary Neuronal Cultures

Primary hippocampal neurons were cultured from the brains of embryo at gestational day 16 (E16). The hippocampi were isolated and treated with 2.5% trypsin solution (Sigma-Aldrich, T4674) for 15 min at 37° C. The cells were washed 3 times with Hank's Balanced Salt Solution (HBSS, Gibco, 14025-092) and mechanically dissociated by pipetting, using glass Pasteur pipettes with 3 different opening sizes (3, 2 and 1 mm). Then, they were counted with the viability dye trypan blue (ThermoFisher Scientific, 15250061), using a hemocytometer, and resuspended in Neurohasal media (Gibco, 21103-049) containing L-glutamine, B27, N2, penicillin and streptomycin. The cells were plated at a density of 60,000 cells per well, respectively, in a 12-well plate on coverslips previously coated with Poly-L-Lysine (Sigma Aldrich, P9155). Cells were cultured for 21 days, and 50% of media was changed every three days.

iPSC-Derived Neuronal Cultures.

Generation of iPSC lines MPS III patient fibroblast lines were obtained from the Coriell Institute for Medical Research (NJ, USA), or from the hospitals were the patients were diagnosed/followed with the informed consent of patient's families. The fibroblasts were propagated in Dulbecco's Modified Eagle Medium (DMEM, ThermoFisher) with 10% fetal bovine serum (FBS) and 1% Antibiotic-Antimycotic (15240062, ThermoFisher) and tested for mycoplasma. The cells were further reprogrammed into iPSCs at the CHUSJ iPSC Platform using a non-integrating CytoTune-Sendai viral reprograming kit (A16517, Thermo Fisher Scientific, MA, USA) according to the manufacture's protocol. Two colonies for each iPSC line were used for further proliferation, iPSCs were expanded and maintained on six-well plates coated with Matrigel mTeSR™ Plus medium at 37° C., in 5% CO₂/5% O₂ atmosphere following the medium manufacturer's protocol. At 60-80% confluency the cells were passaged using the dissociation agent Accutase and plated in mTeSR™ Plus medium containing 10 μM RI (Y27632 ROCK inhibitor, Selleckchem). The following day, the medium was replaced by fresh mTeSR™ Plus medium without RI.

Induction of cortical NPC and cortical neurons iPSCs were differentiated into cortical forebrain committed neural precursor cells (NPCs) by dual SMAD inhibition by passaging iPSCs over to poly-L-ornithine (PO)/laminin coated dishes. NPC induction was performed in a monolayer with the cortical neuronal induction media with FGF-8 used instead of FGFb-2. Eighty percent of media was changed every 2 days. After induction for 3 weeks the cells were analyzed by ICH for the presence of neuronal markers PAX6, and TUBB3, confirmation of disease-specific enzymatic deficiencies and lysosomal storage phenotype (increased size of LAMP2+ puncta by ICH).

Neuronal Differentiation NPCs were differentiated into cortical neurons as follows. First, NPCs were passage into PO/laminin coated plates in a 1/1 mixture of DMEMF-12/neurobasal (NB) media containing B27, N2, NEAA, BDNF, GDNF, Laminin, dbCAMP, Compound E and TGF-B3 containing 2 μM RI. The following day, media was changed for a 100% NB media with containing the above components. Neurons were then cultured for up to 4 weeks until fully differentiated, in the presence or absence of 10 μM AVP6.

### Whole Cell Recordings in Acute Hippocampal Slices.

Acute hippocampal slices were prepared as follows. Briefly, animals were anaesthetized deeply with isoflurane and decapitated. The brain was dissected carefully and transferred rapidly into an ice-cold (0-4° C.) solution containing the following (in mM): 250 sucrose, 2 KCl, 1.25 NaH₂PO₄, 26 NaHCO₃, 7 MgSO₄, 0.5 CaCl₂ and 10 glucose, pH 7.4. The solution was oxygenated continuously with 95% O₂ and 5% CO₂, 330-340 mOsm/L. Transverse hippocampal slices (thickness, 300 μm) were cut using a vibratome (VT1000S; Leica Microsystems), transferred to a bath at room temperature (23° C.) with standard ACSF at pH 7.4 containing the following (in mM): 126 NaCl, 3 KCl, 1 NaH₂PO⁴, 25 NaHCO₃, 2 MgSO₄, 2 CaCl₂), 10 glucose, continuously saturated with 95% 02 and 5% CO₂ and allowed to recover for 1 h. During the experiments, slices were transferred to the recording chamber at physiological temperature (30-33° C.) continuously perfused with standard ACSF, as described above, at 2 ml/min. Pyramidal CA1 neurons from the hippocampus were identified visually using a 40× water immersion objective. Whole-cell patch-clamp recordings were obtained from single cells in voltage- or current-clamp mode and only 1 cell per slice was recorded to enable post-hoc identification and immunohistochemical processing. Recording pipettes (4-6 MΩ) were filled with a K-gluconate based solution for voltage-clamp recordings (in mM): 130 K-gluconate, 10 KCl, 5 diNa-phosphocreatine. 10 HEPES, 2.5 MgCl₂, 0.5CaCl₂,1 EGTA, 3 ATP-Tris, 0.4 GTP-Li, 0.3% biocytin, pH 7.2-7.4, 280-290 mOsm/L.

After obtaining whole cell configuration, passive membrane properties were monitored for 5 min and current clamp recordings were done to measure action potential characteristics. Slices were then perfused with 0.5 μM TTX (to isolate miniature events) for 3 mins before commencing voltage clamp recordings. Cells were voltage clamped at −70 mV for mEPSCs recording and then held at 0 mV (calculated from the reversal potential of Cl) for mIPSCs recording. Data acquisition (filtered at 2-3 kHz and digitized at 15 kHz; Digidata 1440A, Molecular Devices, CA, USA) was performed using the Axopatch 200B amplifier and the Clampex 10.6 software (Molecular Devices). Both mEPSCs and mIPSCs were recorded for 7 min and a running template on a stable baseline (minimum of 30 events) was used for the analysis of miniature events on MiniAnalysis. Clampfit 10.2 software was used for analysis of action potential characteristics and other passive membrane properties.

For some experiments, to verify that all mEPSCs are blocked, slices were perfused with 5 μM DNQX (6,7-dinitroquinoxaline-2,3-dione) and 50 μM AP5 in addition to the TTX while recording mEPSCs at −70 mV after addition of ci-amino-3-hydroxy-5-methyl-4-isoxazolepropionic acid (AMPA) and N-methyl-D-aspartate receptor (NMDAR) blockers. Similarly, for some experiments, slices were perfused with 100 μM BMI (bicuculline methiodide) and 50 μM AP5 in addition to the TTX in the ACSF to verify that all mIPSCS are blocked at 0 mV.

Real-Time qPCR.

RNA was isolated from snap-frozen brain, kidney and liver tissues using the TRIzol reagent (Invitrogen) and reverse-transcribed using the iScript™ Reverse Transcription Supermix (Bio RAD #1708840) according to the manufacturer's protocol, qPCR was performed using a LightCycler® 96 Instrument (Roche) and SsoFast™ EvaGreen® Supermix with Low ROX (Bio RAD #1725211) according to the manufacturer's protocol. RLP32 mRNA was used as a reference control.

### Immunohistochemistry

Mouse brains were collected from animals perfused with 4% PFA in PBS and post-fixed in 4% PFA in PBS overnight. Brains were cryopreserved in 30% sucrose for 2 days at 4° C., embedded in Tissue-Tek® OCT Compound and stored at −80° C. Brains were cut in 40 μm-thick sections and stored in cryopreservation buffer (0.05 M sodium phosphate buffer pH 7.4, 15% sucrose, 40% ethylene glycol) at −20° C. pending immunohistochemistry. Mouse brain sections were washed 3 times with PBS and permeabilized/blocked by incubating in 5% bovine serum albumin (BSA), 0.3% Triton X-100 in PBS for 1 h at room temperature. Incubation with primary antibodies, diluted in 1% BSA, 0.3% Triton X-100 in PBS, was performed overnight at 4° C. The antibodies used in the study and their working concentrations are shown in Table 1:

The mouse brain sections were washed 3 times with PBS and counterstained with Alexa Fluor-labeled secondary antibodies (dilution 1:400) for 2 h at room temperature. After washing 3 times with PBS, the mouse brain sections were treated with TrueBlack® Lipofuscin Autofluorescence Quencher (Biotium, 23007, dilution 1:10) for 1 min, and then again washed 3 times with PBS. The slides were mounted with Prolong Gold Antifade mounting reagent with DAPI (Invitrogen, P36935) and analyzed using Leica DM 5500 Q upright confocal microscope (10×, 40×, and 63× oil objective, N.A. 1.4). Images were processed and quantified using ImageJ 1.50i software (National Institutes of Health. Bethesda, MD, USA) in a blinded fashion. Panels were assembled with Adobe Photoshop.

### Immunocytochemistry

Cultured mouse neurons at day in vitro (DIV) 21 or iPSC-derived neurons at D1V21 and DIV28 were fixed in 4% paraformaldehyde and 4% sucrose solution in PBS, pH 7.4, for 20 min. The cells were permeabilized with 0.1% Triton-X100 in PBS for 5 min, and non-specific binding sites were blocked with 5% BSA (Wisent) in PBS for 2 h and then, incubated overnight at 4° C. with primary antibodies in 1% BSA in PBS (see Table 1 for the source of antibodies and their dilutions). On the following day, neurons were washed 3 times with 1% BSA in PBS and labeled with Alexa Fluor 488- or Alexa Fluor-555-conjugated goat anti-rabbit or Alexa Fluor 633-anti-mouse IgG (1:1000, all from Thermo Fisher Scientific) for one hour at room temperature. Coverslips were washed 3 times again in PBS and mounted on slides using ProLong Gold mounting medium, containing 4′,6-dianiidino-2-phenylindole (DAPI; Invitrogen, Cat #P36935), and analyzed by a Leica SP8-DSL or Leica TCS SPE confocal microscopes (×63 glycerol immersion objectives, N.A. 1.4). Images were processed with Leica Application Suite X (LAS-X) software or Photoshop 2021 (Adobe) and quantified using Fiji-ImageJ 1.50i software (National Institutes of Health, Bethesda, MD, USA). Analysis of images was performed with summation of 9-10 z-stacks separated by 0.5 μm. Soma or axon areas were defined by TUBB3, NEUN, or NF-M staining and, within this area, the appropriate markers were measured establishing a threshold. To obtain LAMP2+ area per neuron, NEUN was used as reference area of the neuron and the image was measured for LAMP2+ puncta while removing background threshold. Quantification was blinded and performed in at least 3 different experiments.

### Western Blot

The cerebral cortical tissues were homogenized in five volumes of RIPA lysis buffer (50 mM Tris-HCl pH 7.4, 150 mM NaCl, 1% NP-40, 0.25% sodium deoxycholate, 0.1% SDS, 2 mM EDTA, 1 mM PMSF), containing protease and phosphate inhibitor cocktails (Sigma, cat#4693132001 and 4906837001), using a Dounce homogenizer. The homogenates were kept on ice for 30 min and centrifuged at 13,000 g at 4° C. for 25 min. The supernatant was centrifuged again at 13,000 g for 15 min, the protein concentration in resulting lysates was measured, and 20 μg of protein from each sample was separated by SDS-PAGE on 4-20% precast polyacrylamide gel (Bio-Rad, 4561096). Western blot analyses were performed according to standard protocols using Anti-BDNF and α-tubulin (1:2000, mouse, DSHB) antibodies. Equal protein loading was confirmed by Ponceau S staining and normalized for tubulin immunoreactive band. Detected bands were quantified using ImageJ 1.50i software (National Institutes of Health, Bethesda, MD, USA).

### Analysis of Glycosaminoglycans by LC-MS/MS

Analysis of brain glycans was conducted as follows. Briefly, 30-50 mg of mouse brain tissues were homogenized in ice-cold acetone and centrifuged at 12,000×g for 30 mM at 4° C. The pellets were dried, resuspended in 0.5 N NaOH and incubated for 2 h at 50° C. Then the pH of the samples was neutralized with 1 N HCl, and NaCl was added to the reaction mix in a final concentration of 3 M. After centrifugation at 10,000×g for 5 mM at room temperature, the supernatants were collected and acidified using 1 N HCl. Following another centrifugation at 10,000×g for 5 min at room temperature, the supernatants were collected and neutralized with 1 N NaOH to a pH of 7.0. The samples were diluted at a ratio of 1:2 with 1.3% potassium acetate in absolute ethanol and centrifuged at 12,000×g and 4° C. for 30 min. The pellets were washed with cold 80% ethanol, dried at room temperature, and dissolved in 50 mM Tris-HCl buffer. The samples were further filtered using AcroPrep™ Advance 96-Well Filter Plates with Ultrafiltration Omega 10 K membrane filters (PALL Corporation, USA) and digested with chondroitinase B, heparitinase, and keratanase II, overnight at 37° C. The samples were analysed by mass spectrometry using a 6460 Triple Quad instrument (Agilent technologies) using Hypercarb columns.

### AVP6 Treatment

Starting from 3 weeks of age, WT C57B16 and homozygous Hgsnat^{P304L} male and female mice were randomly divided in treatment and control groups (6-13 mice/sex/genotype/treatment; see Table I). The control group was daily administered with saline (5 μL to each nostril), while for the treatment group, saline was supplemented with 125 □g of AVP6/mL, which would result in a dose of approximately 50 μg/kg BW/day. The peptide formulation was prepared once, aliquoted and kept frozen at −80° C. until use. At 4 months, all mice were studied by EPM, OF, YM and NOR behavioral tests. Administration of the drug or saline was continued through the days on which the assays were conducted. Then approximately at 5 months 4-5 mice in each group were sacrificed. Their blood plasma was collected, and their tissues were either snap-frozen or fixed and cryopreserved to analyze CNS pathology as described above. For the remaining mice, treatment was continued and their behaviour was studied again at the age of 6 months using OF. NOR and YM tests. Starting from the age of 8 months, Hgsnat^{P304L} treated and untreated mice were daily studied for the signs of urinary retention. When such signs were detected, the mice were studied by ERG and sacrificed within 1-2 days. Finally, the remaining treated and untreated WT mice were studied by ERG and sacrificed at the end of the study, approximately at 10 months of age.

### Statistical Analysis

Statistical analyses were performed using Prism GraphPad 9.0.0. software (GraphPad Software San Diego, CA). The normality for all data was checked using the D'Agostino & Pearson omnibus normality test. Significance of the difference was determined using t-test (normal distribution) or Mann-Whitney test, when comparing two groups. One-way ANOVA test followed by Tukey's multiple comparison test (normal distribution) or Kruskal-Wallis test followed by Dunn's multiple comparisons test were used when comparing more than two groups. Two-way ANOVA followed by Bonferroni post hoc test was used for two-factor analysis. A P-value of 0.05 or less was considered significant.

### Results

### 1. AVP6 Restores Glutamatergic Synaptic Transmission in MPS IIIC Mice

This example tested AVP6's effect on synaptic signaling in acute hippocampal slices of MPS IIIC (both Hgsnat^{Geo} and Hgsnat^{P304L}) mice. To characterize synaptic neurotransmission, we performed whole-cell patch-clamp recordings on acute slices from Hgsnat^{P304L}, Hgsnat-Geo and WT mice at P14-20 and P45-60. At both timepoints the amplitudes of miniature excitatory postsynaptic currents mEPSC were significantly reduced in Hgsnat-Geo and Hgsnat^{P304L} mice as compared with WT mice (FIG. 15A-D). AVP6 is labeled as ACTH(4-7)PGP in these and following figures.

Importantly, for both Hgsnat-Geo and Hgsnat^{P304L} mice, there was an age-dependent (P14-20 vs P45-60) significant decrease in mEPSC amplitudes and a trend for decrease of mEPSC frequencies (FIG. 15E and F). Similar defects were also observed in inhibitory neurotransmission (FIG. 15G and H). Bath application of 10 μM AVP6 recovered deficits in mEPSC amplitude and frequency in both Hgsnat-Geo and Hgsnat^{P304L} strains at P14-20 and at P45-60 (FIG. 15A-D). AVP6 did not increase frequencies or amplitudes of mIPSC in Hgsnat^{Geo} mice at P14-20.

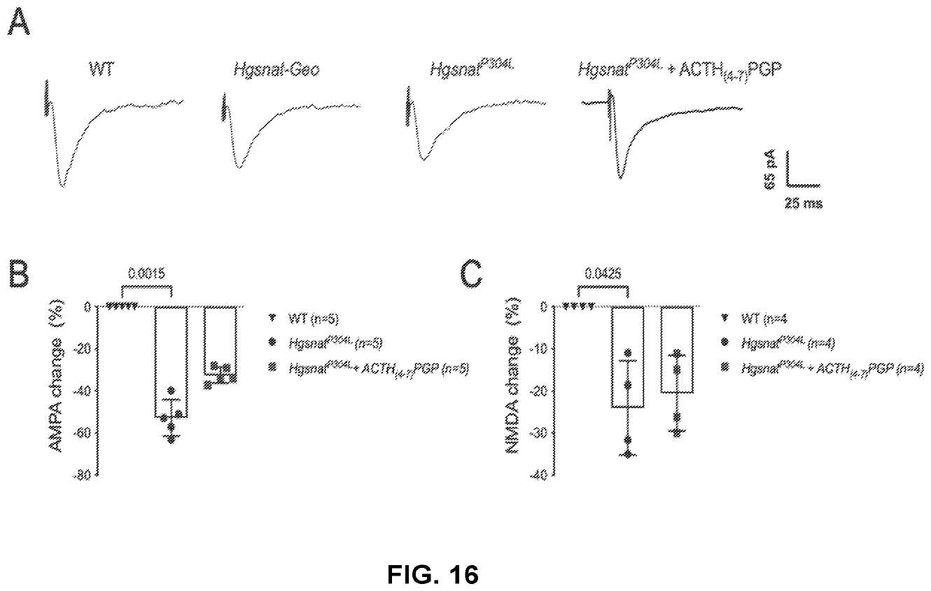

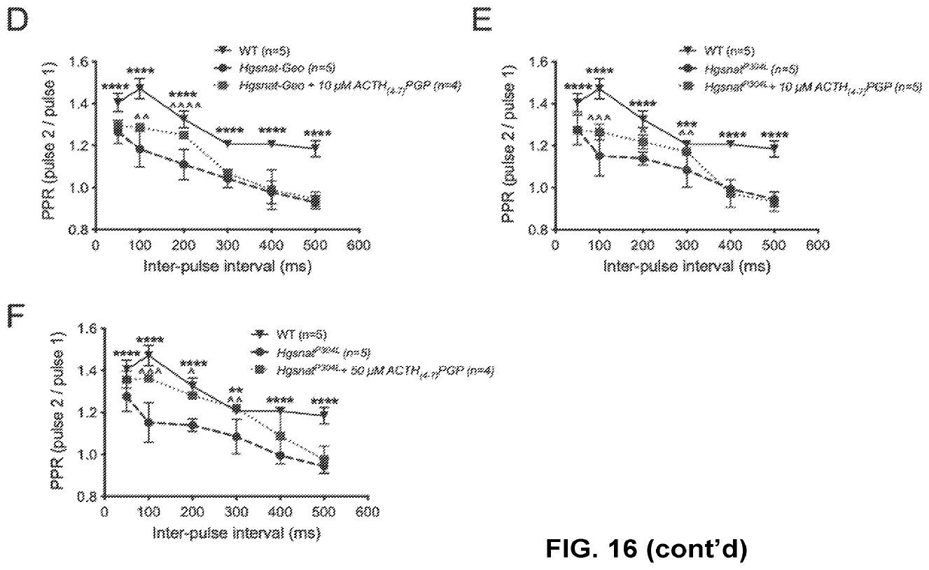

We further studied evoked glutamatergic EPSCs in CA1 pyramidal neurons at P14-20 by stimulating Schaffer Collateral afferents. Upon administering the same intensity of stimulation, both Hgsnat-Geo and Hgsnat^{P304L} mice elicited smaller AMPA and NMDA currents (reflected as significantly reduced AMPA: NMDA ratio) as compared with age-matched WT mice (FIG. 16 A-B). We, next, administered a paired-pulse protocol with decrementing paired-pulse stimuli from 50 ms to 500 ins and found in Hgsnat^{Geo} and Hgsnat^{P304L} mice a significantly lower paired-pulse ratio (PPR) as compared to WT mice measured at the same inter-pulse intervals (IPI) (FIG. 16C-D). This suggests a possibility of a presynaptic AMPA deficit, stemming from improper synaptic vesicle release and recycling, as the readily releasable pool of synaptic vesicles becomes depleted at lower IPI. The effect is aggravated at higher IPI, as both the readily releasable and reserve pools of synaptic vesicles get progressively emptied.

We tested whether bath application of 10 μM AVP6 would increase EPSC, evoked by stimulating Schaffer collaterals in Hgsnat^{P304L} mice, and found that the drug partially recovered deficits in AMPA currents but not in NMDA currents. AVP6 also partially rescued deficits in the paired pulse stimulation in both Hgsnat-Geo and Hgsnat^{P304L} mice with IPI in the range of 100-300 ms, but not at 400 and 500 ms (FIG. 16E and F). When the drug was tested at higher concentration of 50 μM in the brain slices from Hgsnat^{P304L} mice, PPF deficits were rescued at 100, 200, 300 and 400 ms but not at 500 ms IPI (FIG. 16G). Together, these results demonstrated that AVP6 preferentially rescues deficits in AMPA currents, likely through presynaptic mechanisms by increasing release of synaptic vesicles.

2. AVP6 Increases Reduced Levels of Synaptic Protein Markers in Cultured Neurons from MPS IIIC Mice and in iPSC-Derived Cultured Cortical Neurons of Human MPS IIIA and MPS IIIC Patients

Earlier examples demonstrated that synaptic marker, SYN1+ puncta, and the markers of the glutamatergic synapse, VGLUT1+ puncta in juxtaposition with PSD-95+ puncta, were reduced in cultured hippocampal neurons from Hgsnat-Geo mice. The same markers, as well as the markers of the inhibitory synapse VGAT+ puncta in juxtaposition with Gephyrin+ puncta, were also reduced in cultured hippocampal neurons from Hgsnat^{P304L} mice. In order to test whether AVP6 is capable of restoring these deficits, we established embryonic cultures of hippocampal neurons from Hgsnat^{P304L} mice. AVP6 at a final concentration of 10 μM was added to the culture media when the neurons were plated and, further, every 3 days when 50% of the media was changed. At 21 days in vitro (DIV2), neurons were fixed and analyzed by immunohistochemistry using markers of dendrites (MAP2), axons (medium chain of neurofilament protein, NF-M), and synapse (SYN1, VGLUT, and PSD-95). We also analyzed the levels of BDNF to test if this protein was deficient in the hippocampal neurons from Hgsnat^{P304L} mice and whether it was increased by the treatment with AVP6 peptide. The numbers of puncta positive for the above markers were counted in 20 μm-long segments of a dendrite or an axon, 30 μm away from the neuronal soma.

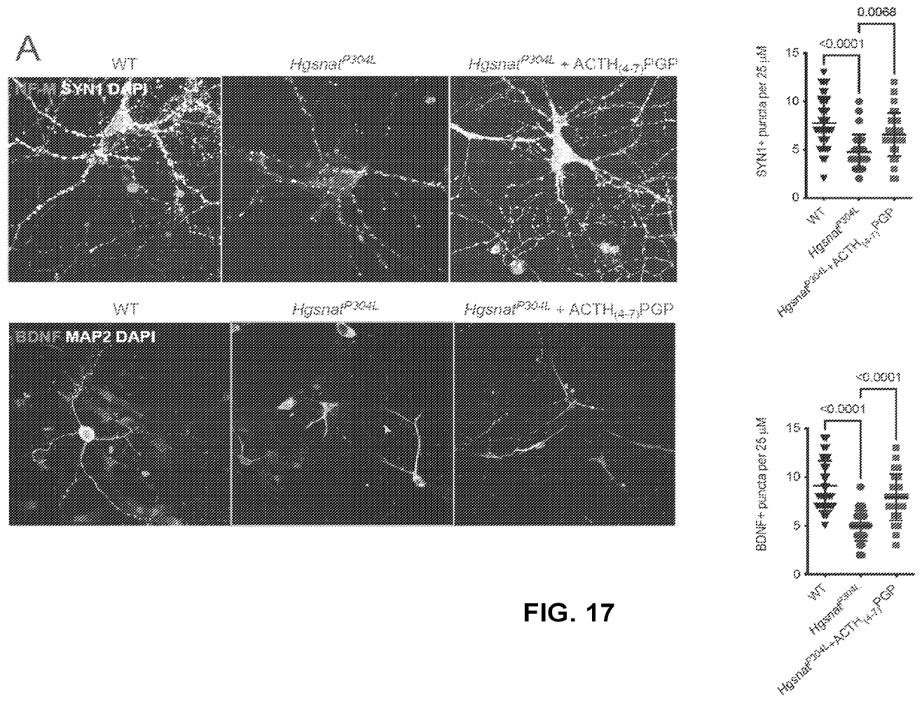

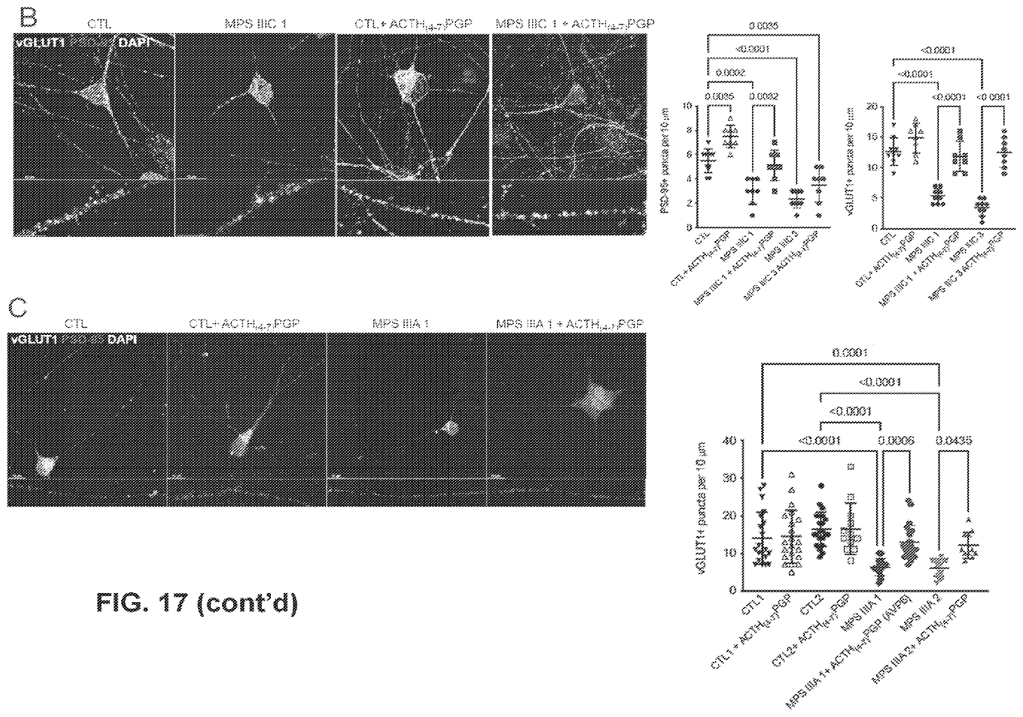

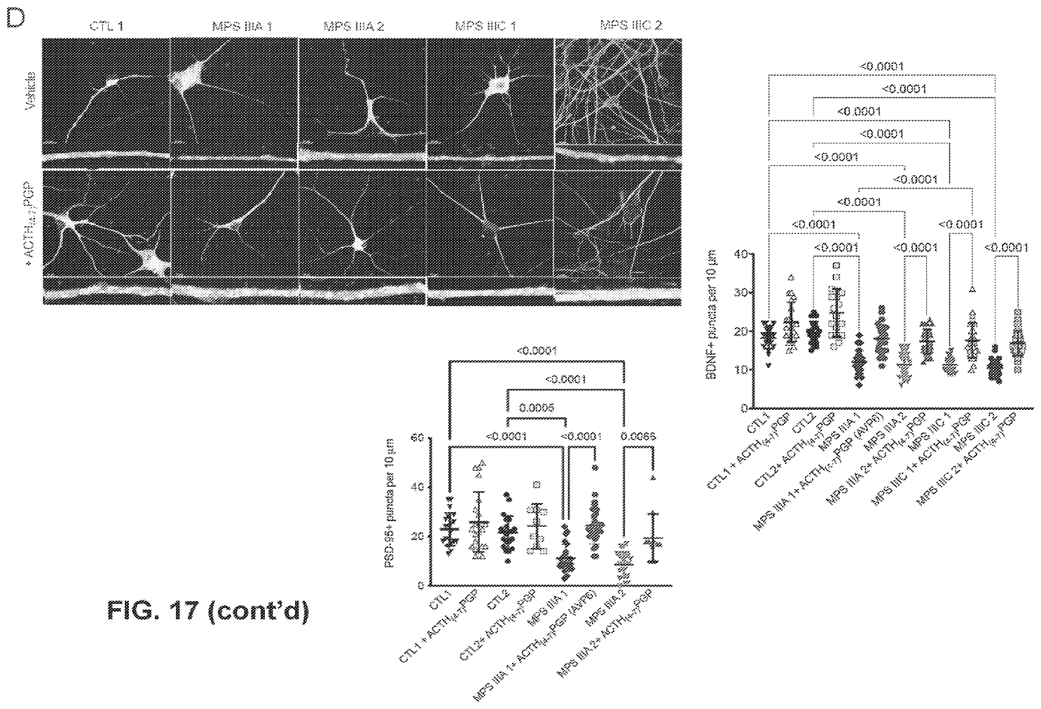

Our results indicate that primary neuronal cultures from Hgsnat^{P304L} mice show drastic reduction of BDNF and SYN1, while the treatment with AVP6 rescues deficit of both proteins (FIG. 17A).

To test whether the effect of the peptide can be recapitulated in neurons of human MPS IIIC patients and patients with other subtypes of MPS III, we have generated iPSC lines from available skin fibroblast lines received from cell depositories or obtained with consent of families. The fibroblasts were reprogrammed using the Sendai virus manufactured by Life Technologies. All iPSCs lines had a normal karyotype, were positive for pluripotency markers TRA-1-60 and SOX2, and demonstrated ability to differentiate in vitro into the three germ layer cells (Nestin+/PAX6+ ectoderm, SMA+ mesoderm and SOX17 (CXCR4)+ endoderm).

After confirmation of the primary enzymatic defect (HGHS for MPS IIIA, NAGLU for MPS IIIB, HGSNAT for MPS IIIC and GNA for MPS IIID), the iPSCs were differentiated into forebrain committed neural precursor cells (NPC) by dual SMAD inhibition. NPC were induced in neuronal induction media (DMEM/F12) for 3 weeks and analyzed by immunocytochemistry to confirm expression of the neuronal markers, NeuN, axonal β-tubulin III (clone TUJ1) and SYN1. Increased size of LAMP2+ puncta and high levels of total β-hexosaminidase activity were detected in the NPC lines from MPS IIIC patients as compared with cells from healthy controls suggesting the lysosomal storage phenotype and increased lysosomal biogenesis. As for iPSC, primary HGHS, NAGLU, HGSNAT or GNA deficiency in generated NPC lines was confirmed by measuring enzyme activity in cell homogenates.

Subsequently, NPC were differentiated into the cortical neurons by culturing in 1/1 mixture of DMEMF-12/neurobasal (NB) media containing B27, N2, NEAA, BDNF, GDNF, Laminin, dbCAMP, Compound E and TGF-B3. Neurons were further cultured for 4 weeks until they were fully differentiated, fixed and stained for SYN1, VGLUT1, PSD-95, BDNF, and LAMP2. Similarly to NPC, iPSC-derived neurons of MPS III patients showed a significant increase in LAMP2 staining indicating lysosomal storage and increased lysosomal biogenesis.

Importantly, MPS III iPSC derived neurons showed significantly reduced levels of BDNF+, VGLUT1+ and PSD-95+ puncta suggesting that deficiency of protein markers of glutamatergic synapse observed in cultured neurons from Hgsnat^{P304L} mice, is recapitulated in human MPS IIIA and MPS IIIC cells (FIG. 17B, C and D). Importantly levels of BDNF+, VGLUT1+ and PSD-95+ puncta were significantly increased in the neurons treated in culture with 10 μM AVP6 (FIG. 17B, C and D).

Altogether, our data demonstrate that AVP6 rescues reduced levels of the protein markers of the glutamatergic synapse in cultured neurons. Together with the ability of the peptide to induce miniature and evoked excitatory currents at the presynaptic side, these results suggest that the drug rescues glutamatergic synaptic deficits in MPS III neurons in vitro and ex vivo.

3. Short-Term Treatment with AVP6 Partially Rescues Neurobehavioral Manifestations and Increases Hippocampal BDNF Levels in Symptomatic MPS IIIC Mice

We further tested if AVP6 can rescue neurobehavioral deficits associated with synaptic dysfunction in MPS IIIC mice. The early phase of the disease in both Hgsnat-Geo and Hgsnat^{P304L} mice manifests with reduced anxiety and hyperactivity. Specifically, in an Open Field (OF) test at both 4 months and 6 months, Hgsnat^{P304L} mice show a significant increase in a total distance traveled, increased time spent at the center of the arena and increased distance traveled in the center of the arena as compared with the WT animals. Earlier examples demonstrated that AVP6 is readily targeted to the brain and exerts the maximal effect on memory and learning within 24 hours after intranasal administration at a dose of 50 μg/kg BW in mice and rats. Thus, 4 and 6-month old Hgsnat^{P311L} and WT mice were studied by OFT 17 hours after intranasal administration of the peptide in a single dose of 50 μg/kg BW (˜5 μl of 125 mg/ml peptide solution in saline per each nostril). Control groups were treated with the same volume of saline.

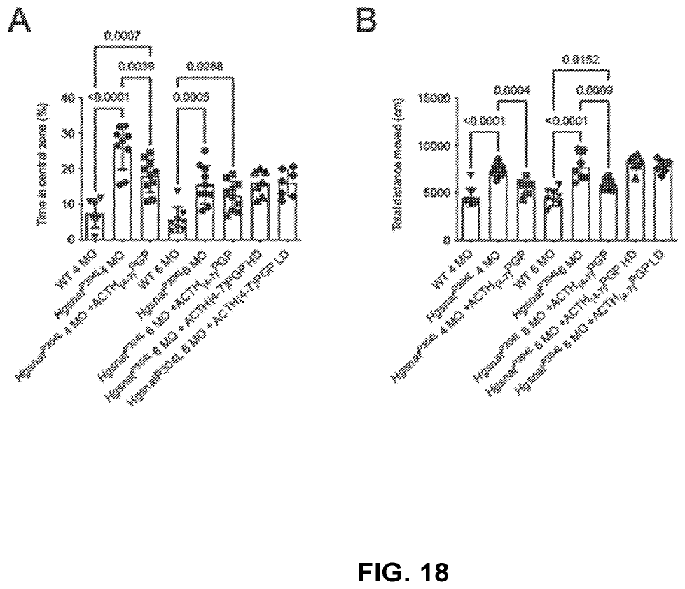

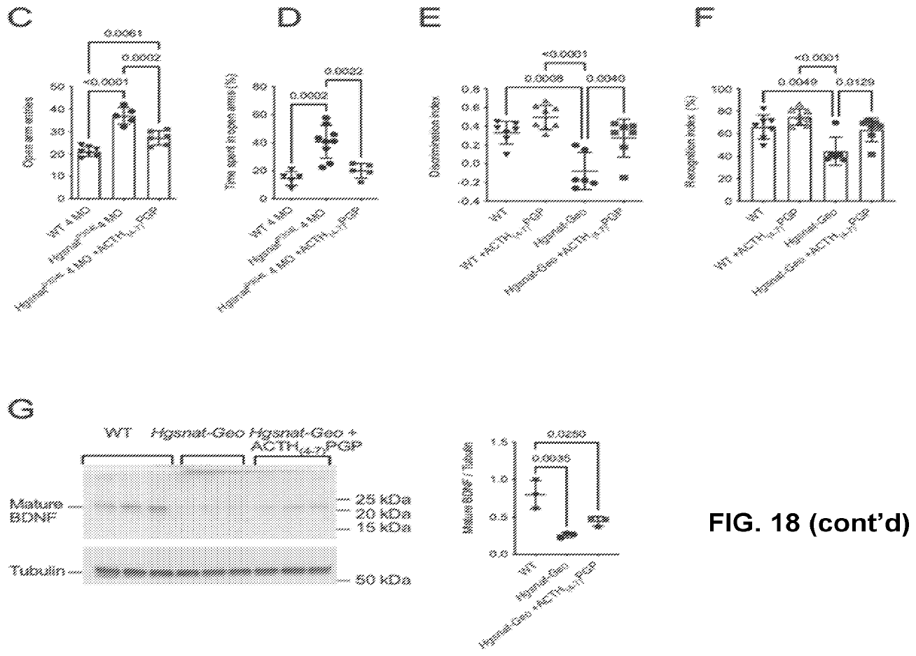

We found that in Hgsnat^{P311L} mice of both ages, treated with AVP6, the total traveled distance was reduced and the distance traveled in the center/time spend in the center increased as compared with untreated mice indicating that the treatment partially reversed the behavioral deficits (FIG. 18A and B). We further tested a lower (10 μg/kg BW) or a higher (500 μg/kg BW) doses of the peptide in 6-month-old Hgsnat^{P304L} mice and found that both failed to rescue hyperactivity or reduced anxiety (FIG. 18C). Ability of the peptide to rescue reduced anxiety was also tested in the Elevated Plus Maze test (EPM) that measures a natural fear of heights reflex of animals. Hgsnat^{P304L} mice at 4 months and Hgsnat-Geo mice at 6 months show significantly reduced fear (increased time spent in open arms and increase in the number of open arm entries) as compared to the WT animals of the corresponding age, while animals treated with a single dose of AVP6 showed a behavior similar to their WT counterparts (FIG. 18D).

The ability of the peptide to improve the memory deficits was tested in the 4-months-old Hgsnat-Geo mice using the Novel Object Recognition (NOR) test that studies working memory of mice by measuring their ability to discriminate a familiar object a novel one. AVP6 was administered intranasally at a daily dose of 50 μg/kg for ten consecutive days before the experimental day. As before, the control groups of Hgsnat-Geo and WT animals were treated with saline. We found that saline-treated Hgsnat-Geo mice showed significantly reduced values of a discrimination index and a recognition index suggesting their reduced ability to recognize the familiar object (FIG. 18E). At the same time, the values of a discrimination index and a recognition index for Hgsnat-Geo mice, that were receiving the peptide were similar to the WT controls suggesting rescue of the short-term memory deficit. The values of a discrimination index and a recognition index for the WT mice treated with AVP6 showed a trend for an increase as compared with the WT mice treated with saline, but the effect was not statistically significant (FIG. 18E). Immediately after the test, mice were sacrificed and the levels of mature BDNF protein were measured in their hippocampi by immunoblot. While Hgsnat-Geo mice treated with saline showed reduced levels of mature BDNF in hippocampus as compared to WT animals, the animals treated for 10 days with AVP6 demonstrated partially restored levels of this protein (FIG. 18F).

At the same time, a single dose of AVP6 17 hours before the test, did not improve behaviour of Hgsnat-Geo mice in NOR test (data not shown) indicating that long-term administration of the peptide is required to rescue memory deficit.

3. Chronic Treatment with AVP6 Delays Neurobehavioral Manifestations and Development of Pathological CNS Changes in the Hgstzat^{P304L} Mice

Encouraged by the results of preliminary studies indicating that intranasal administration of AVP6 can induce brain levels of mature BDNF and partially rescue neurobehavioral deficits in the mouse models of MPS IIIC we conducted a preclinical efficacy study to test whether chronic administration of the peptide can delay clinical and pathological manifestations of the disease. The study was conducted in the Hgsnat^{P304L} strain that shows more aggressive course of the disease as compared with Hgsnat-Geo mice.

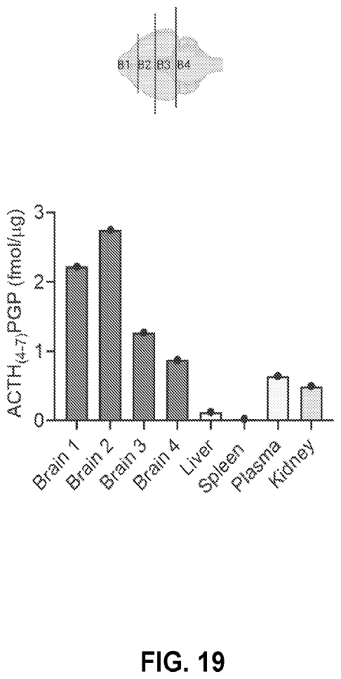

We have selected the invasive intranasal administration, which is potentially ideal for clinical application, as the primary delivery route. Peptide pharmaco-kinetics was determined by evaluation of AVP6 levels in CNS, blood and peripheral tissues at different time points (1-48 h) after intranasal delivery in the WT mice. The entire mouse brain was cut in four sections, rostral to caudal and assessed for biodistribution of AVP6 by targeted LC-MS/MS using parallel reaction monitoring. For quantification, samples were spiked with isotopically labeled (Phe U-¹³C₉; U-¹⁵N) AVP6 peptide as an internal standard. Peptide levels were also measured in peripheral (liver, kidney, spleen) tissues and in blood to provide insights into peptide biodistribution and degradation rates. These experiments demonstrated that 1 h after intranasal administration (10 □l of 50 mM AVP6) the concentration of the peptide in the brain (2.8-0.9 fmol/□g) is much higher than in plasma or visceral organs and exceeds the concentration estimated to be effective for restoring the neurotransmission (FIG. 19). The level of the peptide in the brain remained above the estimated acting concentration for 17 h after administration. We thus have chosen a daily administration as the drug regimen.

WT, and Hgsnat^{P304L} mice were randomly assigned to the treatment and control groups. The cohort size (18 mice/sex/treatment) was calculated based on mean variability of replicates in previous behavioral tests in Hgsnat^{P304L} mice to detect a ˜40% difference between means (power=0.8). Treatment was started at weaning (P21) which corresponds to neurodevelopmental human age of 3 years, the time of disease onset for majority of Sanfilippo patients. Since most patients are diagnosed post-symptomatically, this age would most likely become the treatment starting point for the most of patients. Although Hgsnat^{P304L} mice at P21 do not show behavioral alterations, their CA1 pyramidal neurons show synaptic deficits at the electrophysiological level and significantly reduced density of dendritic spines at this age. To test if chronic administration of the peptide results in major metabolic changes, the mouse body weight was measured weekly. No difference in body weight and body weight gain was detected between the treated and untreated Hgsnat^{P304L} or WT mice.

Since phenotypic differences between WT and Hgsnat^{P304L} mice are already pronounced at 16 weeks, mice were treated between 6 and 16 weeks, at which point their behavior was assessed as before by OF (anxiety, fear and hyperactivity), EPM (anxiety, fear), YM and NOR (memory) tests.

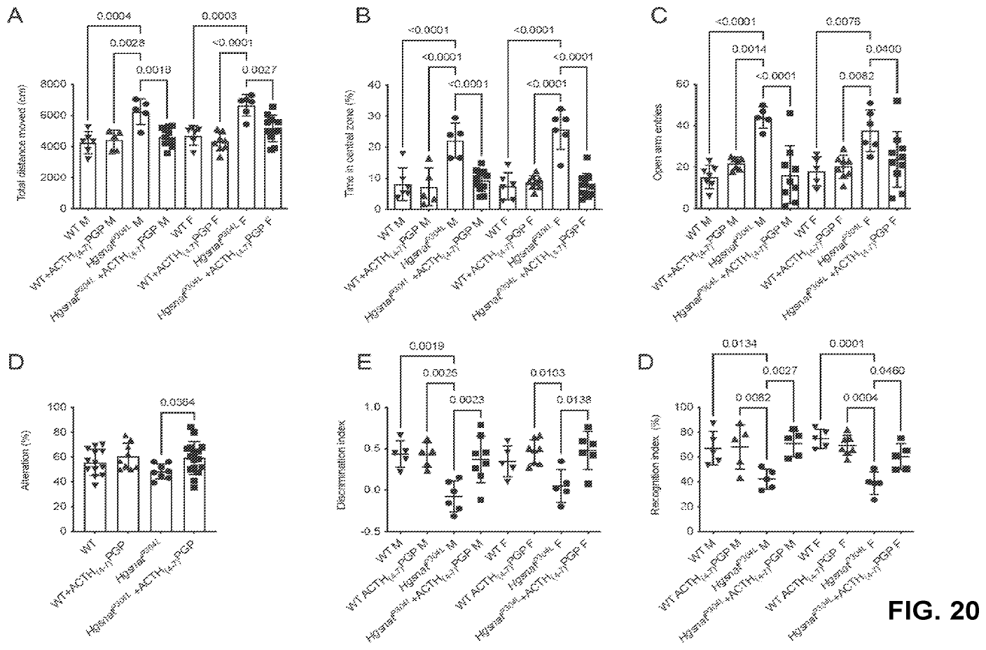

When analyzed by OF test at 4 months, both male and female Hgsnat^{P304L} mice treated with saline showed significantly increased hyperactivity (increase in the total distance traveled) and reduced anxiety (increased time spent in the center of the arena) as compared to the WT animals (FIG. 20A and B). In contrast, both male and female Hgsnat^{P304L} mice, chronically treated with AVP6, showed absence of these phenotypes (FIG. 20A and B). Importantly, there was no significant difference between male and female mice in their response to the treatment. Also no difference was observed between the female and male WT mice treated with saline and those treated with AVP6.

In similar fashion, female or male Hgsnat^{P304L} mice, treated with saline at 4 months, showed significantly reduced fear when studied by EPM test (increase in the number of open arm entries and increased time spent in the open arms) as compared to the WT animals (FIG. 20C and D).

Both male and female Hgsnat^{P304L} mice, treated daily with AVP6, showed no difference from their WT counterparts in the number of open arm entries and the percentage of time spent in the open arena. As before, there was no sex specific effect in any of the parameters assayed in the elevated plus maze test and no effect of the drug was detected in the WT mice.

The short-term and spatial memory of mice was studied by NOR and YM tests. As before, female and male Hgsnat^{P304L} mice treated with saline, showed a significant reduction in discrimination and recognition indexes, suggesting a short memory deficit, while both male and female Hgsnat^{P304L} mice treated with AVP6, were similar to the WT mice (FIG. 20E and F). There was a trend for reduction of alternation index in the YM test for both female and male saline-treated Hgsnat^{P304L} mice but not for AVP6-treated Hgsnat^{P304L} mice. However, because of a higher variation between individual mice, a significant difference between saline-treated and peptide-treated mice was observed only, when we pooled the data for both sexes together (FIG. 20G). Together, all data demonstrated that daily treatment with AVP6 prevented development of neurobehavioral deficits in the Hgsnat^{P304L} mice at 4 months.

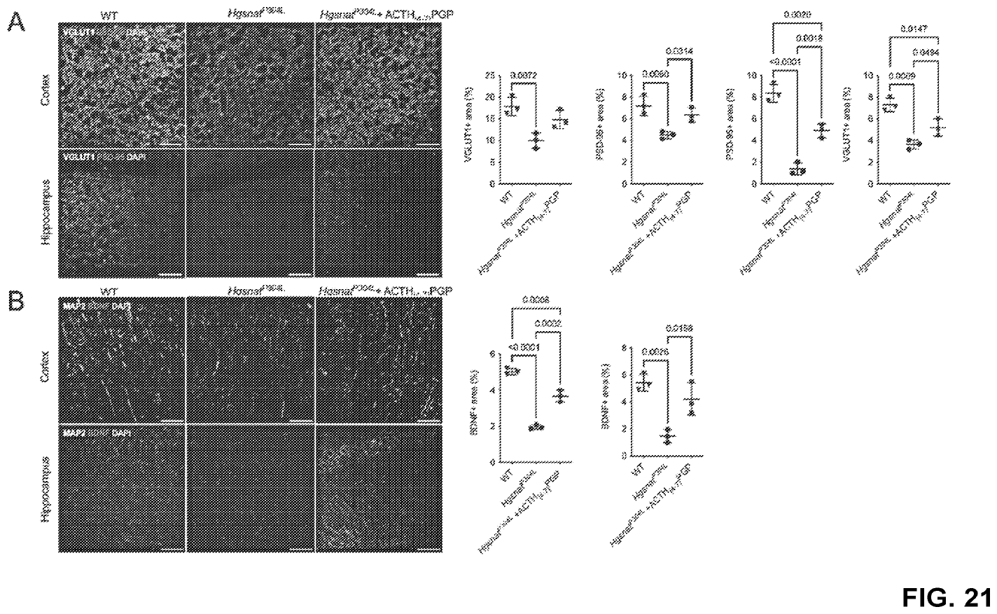

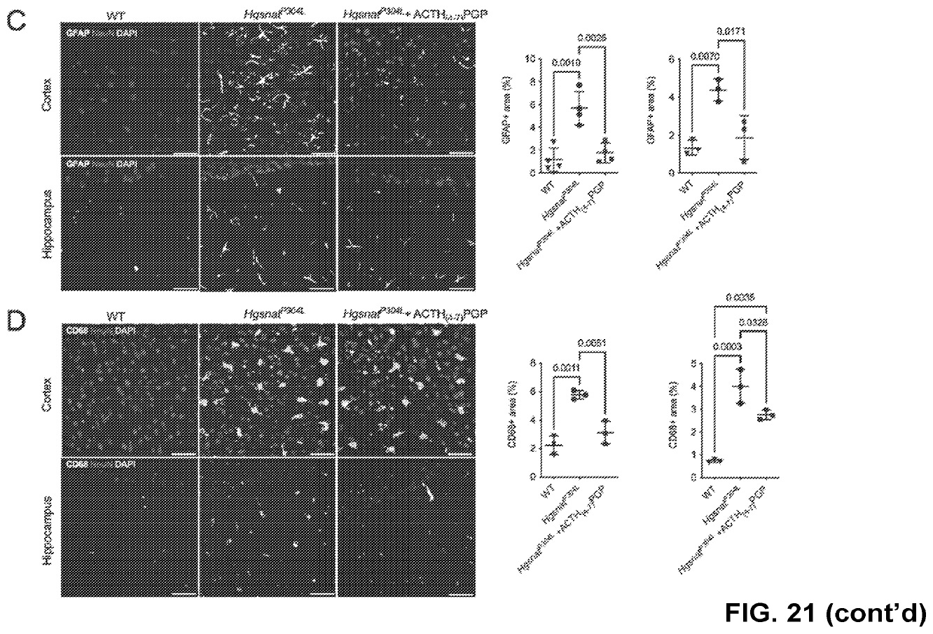

After completion of the behavioural analysis at 4 months, 50% of mice in each group were sacrificed for analysis of CNS pathology and biochemical testing. To study the effect of the treatment on synaptic architecture, we have stained the brain sections for presynaptic (VGLUT1) and postsynaptic (PSD-95, BDNF) markers and quantified their levels in the CA1 area of hippocampi and in layers 3-4 of somatosensory cortex. Our data (FIG. 21A and B) showed that in both areas all markers were significantly reduced in Hgsnat^{P304L} mice treated with saline as compared with saline-treated WT mice. In contrast, the levels of all 4 markers in the brains of the AVP6-treated Hgsnat^{P304L} mice were similar to those in the WT mice.

Unexpectedly, we also observed that the abundance of GFAP+ astrocytes and CD68+ activated microglia were reduced in both brain areas of AVP6-treated Hgsnat^{P304L} mice as compared with saline-treated Hgsnat^{P304L} mice, suggesting that the drug partially blocked the neuroimmune response (FIG. 21C and D). This coincided with reduced expression levels of inflammatory cytokine MIP1α in the brains of AVP6-treated as compared with saline-treated Hgsnat^{P304L} mice (FIG. 21E). At the same time the levels of total β-hexosaminidase activity in the total brain homogenates or the levels and sizes of LAMP2+/HS+ or GM2-ganglioside+ lysosomal puncta in the cortical/hippocampal neurons (not shown) remained similar for the AVP6-treated and with saline-treated Hgsnat^{P304L} mice, suggesting that the treatment did not reduce levels of lysosomal storage and lysosomal biogenesis.

4. Chronic Treatment with AVP6 Prolongs Survival and Ameliorates CNS and Peripheral Tissue Pathology in the Hgsnat^{P304L} Mice at the Terminal Stage of the Disease

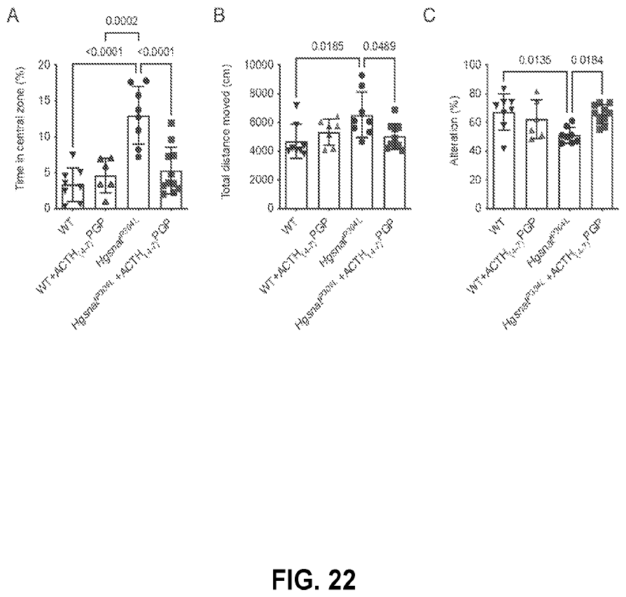

About 50% of mice in all cohorts were continued to be treated with AVP6 or saline for assessment of the peptide effect on the behavioural abnormalities at 6 months of age and survival. Since we did not have enough number of animals to evaluate the effect of the treatment for each sex separately, the behaviour of male and female mice in the peptide-treated and saline-treated groups was analyzed separately and compared. If no difference was observed between sexes in the same group, the results were pooled. The OF test, conducted at 6 months, revealed that reduced anxiety (significantly increased percentage of time spent in the center of the arena) and hyperactivity (increased total distance traveled in the arena) were significantly reduced in Hgsnat^{P304L} mice treated with AVP6 as compared with the Hgsnat^{P304L} mice treated with saline (FIG. 22A). No difference in behaviour was observed between saline-treated and AVP6-treated WT mice. The short-term and spatial memory were evaluated at 6 months of age using the YM test and the NOR test. In the YM test, saline-treated Hgsnat^{P304L} mice at 6 months showed significantly reduced percent of alternation between arms as compared to the saline-treated WT animals while AVP6-treated Hgsnat^{P304L} mice demonstrated alternation similar to that of the WT mice (FIG. 22B).

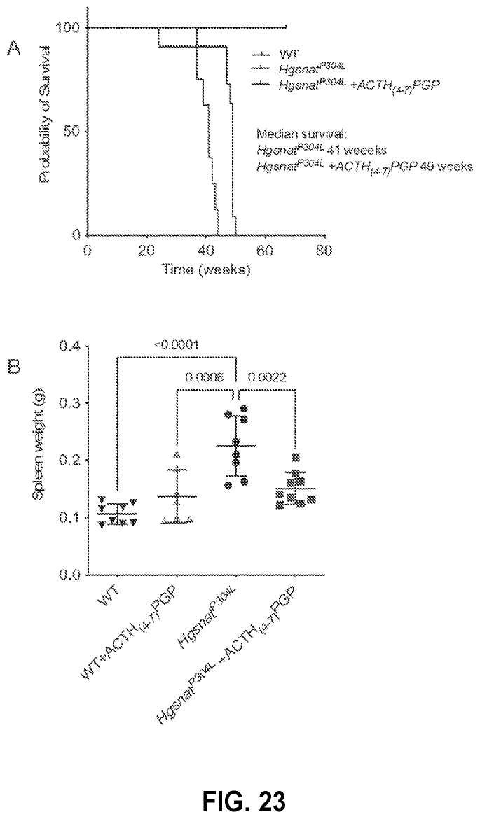

Unlike human MPS III patients, Hgsnat^{P304L} mice at the age of approximately 8 months develop urinary retention resulting in abdominal distension and requiring humane euthanasia. The mechanism underlying this phenotype, observed also in other murine models of neurological MPS, is not completely clear, but it was proposed to be associated with GAG storage and infiltration of immune cells in the epithelium of the urinary tract and bladder. Previously we determined the average survival age of Hgsnat^{P304L} mice as 42 weeks. To test whether the AVP6 treatment delayed development of this phenotype, mice in both treatment and vehicle groups were examined for the signs of urinary retention on a daily basis, starting from the age of 7 months, and immediately sacrificed, when abdominal distension was detected. The WT mice in the treatment and vehicle groups were sacrificed one week after the sacrifice of the last treated Hgsnat^{P304L} mouse. We found that the AVP6-treated Hgsnat^{P304L}, in general, showed a longer survival with the average life span of 49 weeks, which is 8 weeks longer that the survival of saline-treated group (FIG. 23A). When the wet weights of mouse spleen were measured at sacrifice to assess the extent of visceromegaly, we found that the AVP6-treated Hgsnat^{P304L} mice had significantly lower spleen weight that the saline-treated Hgsnat^{P304L} mice despite being, on average, 8 weeks older (FIG. 23B). This suggested that the treatment also reduced inflammatory response in some peripheral tissues.

The terminal CNS pathology was studied using the same set of biomarkers as at 4 months, either by IHC (astrocytosis, microgliosis, synaptic markers SYN1, PSD-95, GLUT1, BDNF) or by biochemical (total β-hexosaminidase activity, expression levels of inflammatory cytokines) assays.

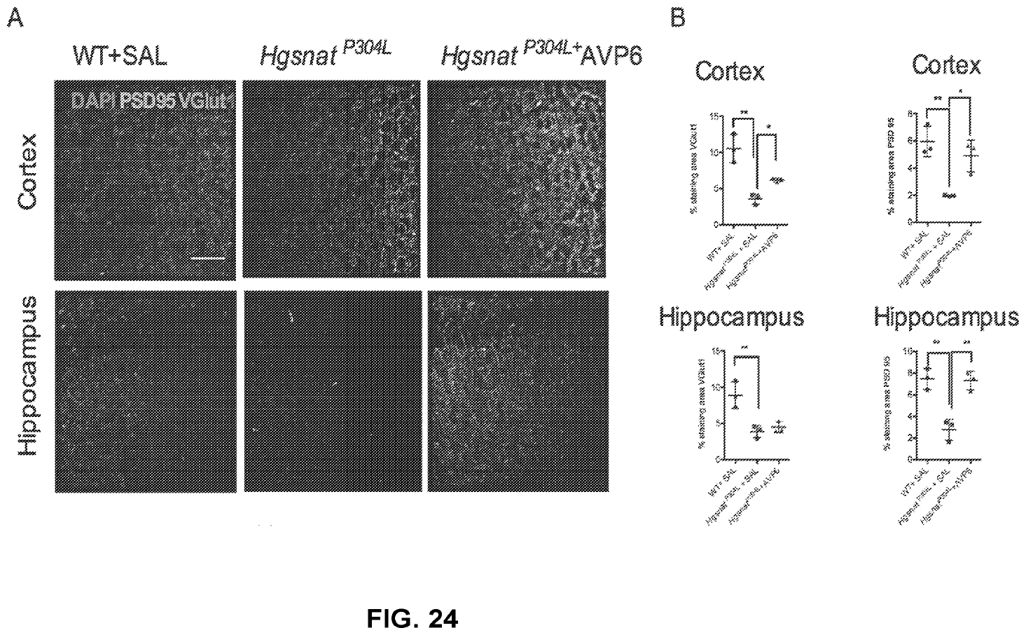

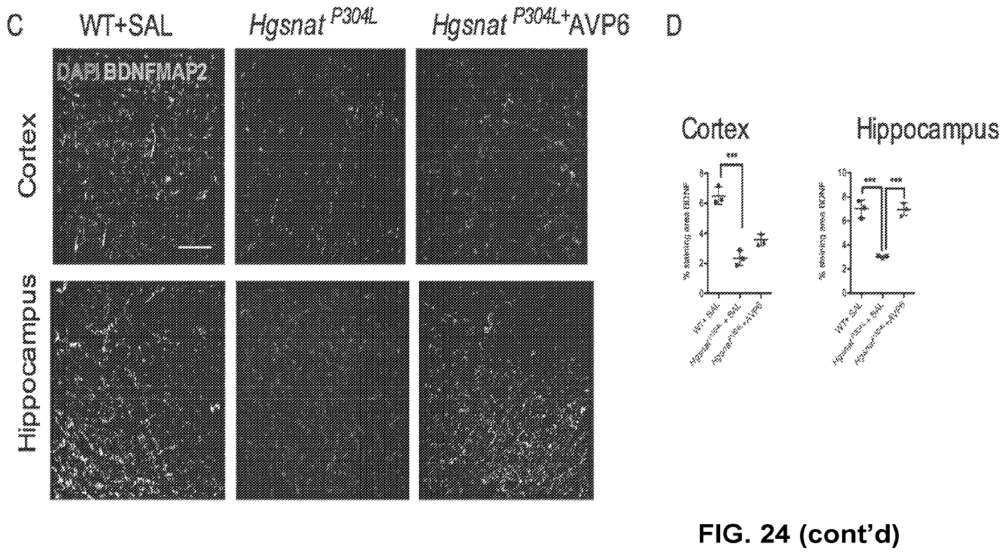

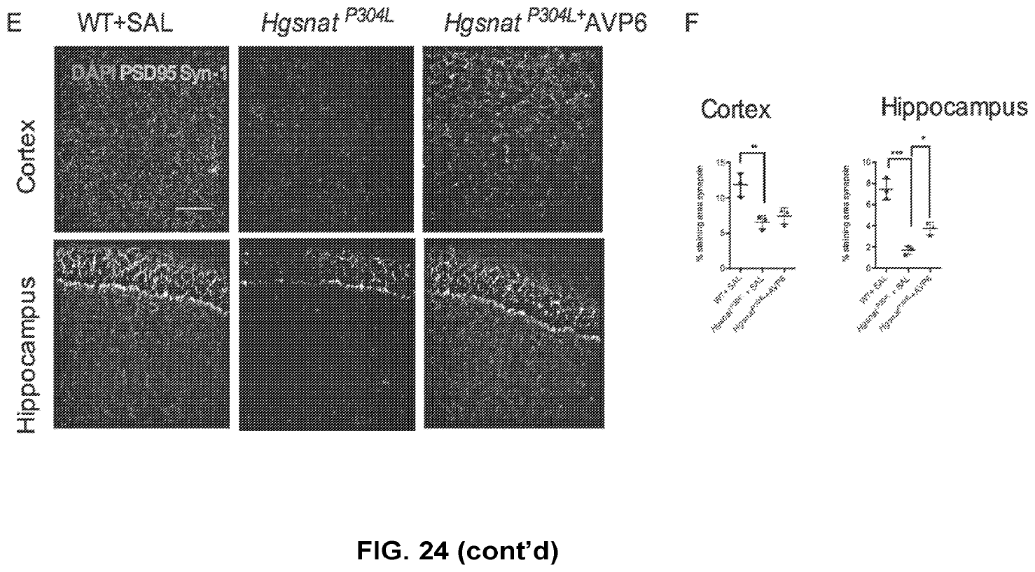

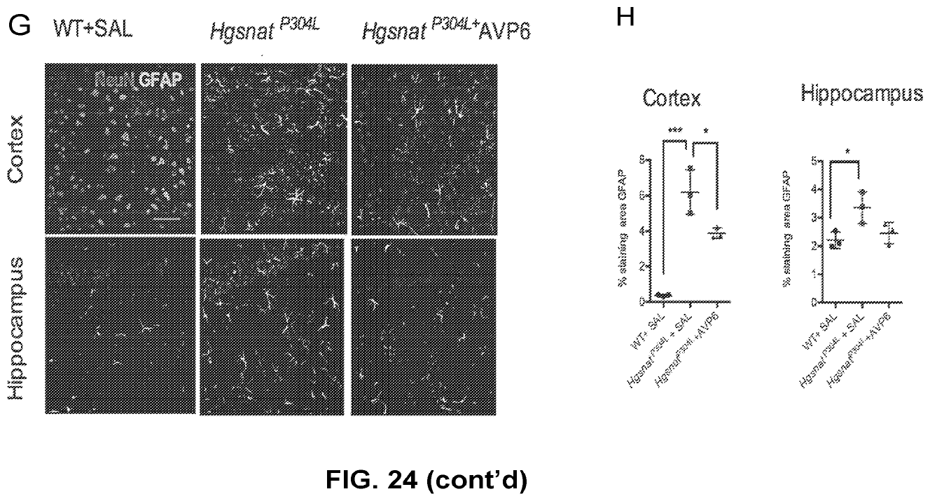

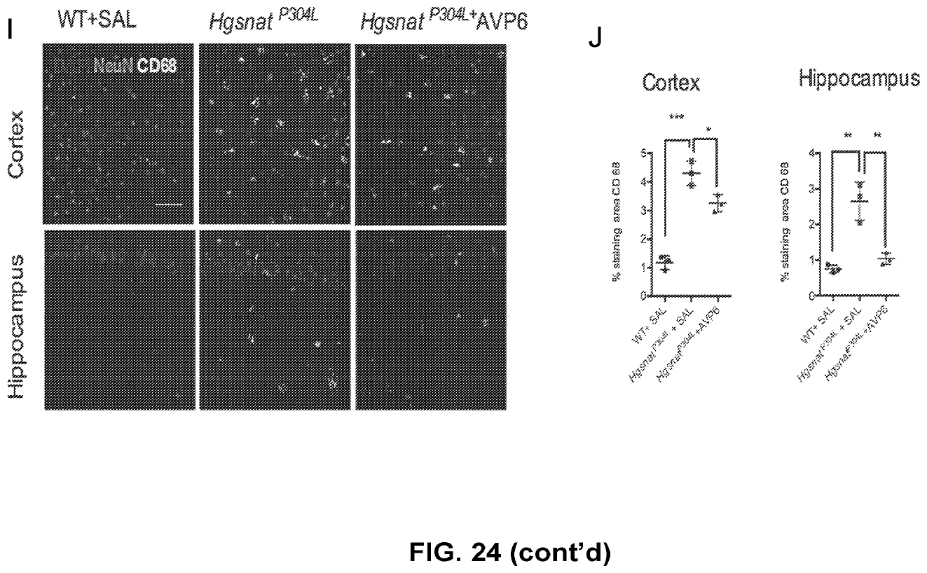

As at the age of 4 months AVP6-treated Hgsbat^{P304L} mice at the age of sacrifice (10-11 months) showed a significantly increased levels of synaptic protein markers as compared with saline-treated Hgsnat^{P304L} mice. However, the effect was different for pre- and post-synaptic proteins. While levels of post-synaptic proteins, PSD-95 and BDNF, were completely recovered and similar to those in the WT mice, the levels of presynaptic proteins, GLUT1 and SYN1, either increased in the AVP6-treated as compared with saline-treated Hgsnat^{P304L} mice, or remained significantly lower than those in WT animals (FIG. 24A-C). Markers of astrocytosis and microgliosis, GFAP and CD68, were significantly reduced in AVP6-treated as compared with saline-treated Hgsnat^{P304L} mice both in the cortex and hippocampus, however their levels in the cortex remained significantly increased as compared with WT mice (FIG. 24D and E).

Total β-hexosaminidase activity measured in brain homogenates although showed a trend for reduction in AVP6-treated as compared with saline-treated Hgsnat^{P304L} mice remained significantly increased as compared with treated or untreated WT mice as was the LAMP2 levels in the brain sections suggesting that treatment does not ameliorate levels of lysosomal storage at the terminal timepoint.

The results of our study demonstrate that AVP6 can ameliorate pathological signs in animal and cellular models of mucopolysaccharidosis IIIA and IIIC.

At the Cellular and Synaptic Levels:

The treatment of cultured primary hippocampal neurons of Hgsnat^{P304L} mice with 10 μM AVP6 added to the culture media rescues reduced levels of synaptic markers VGLUT1, SYN1, PSD-95 and BDNF.

The treatment of cultured iPSC-derived cortical neurons from MPS IIIA and MPS IIIC patients by 10 μM AVP6 added to the culture media rescues reduced levels of synaptic markers VGLUT1, SYN1, PSD-95 and BDNF, demonstrating that the drug acts on human cells affected with different subtypes of the disease.

Bath application of AVP6 rescues deficits in glutamalergic neurotransmission in acute hippocampal slices of Hgsnat^{Geo} and Hgsnat^{P304L} MPS IIIC mice at both P14-20 and P45-60. The peptide preferentially rescues deficits in AMPA currents.

At the behavioral level:

Single intranasal administration of AVP6 to 4-month-old and 6-month-old MPS IIIC Hgsnat^{P304L} mice at a dose of 50 μg/kg BW rescues reduced anxiety and hyperactivity in OF and EPM tests 17 hours after the treatment. Single intranasal administration of AVP6 to Hgsnat-Geo MPS IIIC mice at a dose of 50 μg/kg BW also rescues reduced anxiety in EPM test 17 hours after the treatment.

Ten-day intranasal administration of AVP6 to 4-month-old Hgsnat-Geo MPS IIIC mice at a dose of 50 μg/kg BW rescues impairment of short-term memory in NOR test.

Chronic treatment of Hgsnat^{P304L} mice with AVP6 at a dose of 50 μg/kg BW/day rescues reduced anxiety and hyperactivity in OF and EPM tests at 4 and 6 months.

Chronic treatment of Hgsnat^{P304L} mice with AVP6 in a dose of 50 vg/kg BW/day rescues deficits in spatial and short-term memory in YM and NOR tests at 4 months and partially rescues deficits in spatial and short-term memory in spatial and short-term memory in YM and NOR tests at 6 months.

At the pathophysiological level:

Chronic treatment with AVP6 at a dose of 50 μg/kg BW/day rescues reduced BDNF levels in hippocampal and cortical pyramidal neurons of Hgsnat^{P304L} mice at the age of 5 months and partially rescues them at the age of 8-9 months coinciding with the improvements of memory deficits observed at 4 months and 6 months.

Chronic treatment with AVP6 at a dose of 50 lag/kg BW/day rescues reduced levels of synaptic proteins SYN1, VGLUT1 and PSD-95 in hippocampal and cortical pyramidal neurons of Hgsnat^{P304L} mice and the age of 5 months and partially rescues them at the age of 8-9 months.

Chronic treatment with AVP6 at a dose of 50 μg/kg BW/day normalizes astrocytosis and microgliosis in Hgsnat^{P304L} mice at the age of 5 months and reduces it at the age of 10-11 months, demonstrating, also, that the drug has anti-inflammatory action.

Chronic treatment with AVP6 at a dose of 50 μg/kg BW/day delays the onset of lethal urinary retention by approximately 8 weeks and reduces splenomegaly in Hgsnat^{P304L} mice, demonstrating that the drug also exerts an anti-inflammatory effect in peripheral tissues.

Together, these data demonstrate that AVP6 delays neurological manifestations in MPS IIIC by rescuing glutamatergic neurotransmission and synaptogenesis defects. They also demonstrate that the drug delays immunoinflammatory response in CNS and peripheral tissues and increases longevity.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for treating a neuropathophysiological condition in a patient in need thereof, comprising administering to the patient an effective amount of an agent that increases the biological activity of brain-derived neurotrophic factor (BDNF).

2. The method of claim 1, wherein the patient suffers from a lysosomal storage disorder (LSD).

3. The method of claim 1, wherein the LSD is selected from the group consisting of a lipid storage disorder, a mucopolysaccharidosis, a glycoprotein storage disorder, and a mucolipidosis.

4. The method of claim 3, wherein the LSD is the mucopolysaccharidosis (MPS).

5. The method of claim 4, wherein the mucopolysaccharidosis (MPS) is selected from the group consisting of MPS I, MPS II, MPS III, MPS VII, and MPS IX.

6. The method of claim 5, wherein the MPS is MPS IIIA, MPS IIIB, MPS IIIC, or MPS IIID.

7. The method of claim 1, wherein the neuropathophysiological condition is selected from the group consisting of dementia, aggressive behavior, hyperactivity, seizure, deafness and loss of vision.

8. The method of claim 1, wherein the agent is selected from the group consisting of nootropic peptides, Acetyl L-Carnitine (ALCAR), Alpha-GPC, Alpha-Lipoic Acid (ALA), Aniracetam, Ashwagandha, Artichoke Extract (Luteolin), Bacopa Monnieri, Berberine, Black Seed Oil, Cacao, Caffeine, Cat's Claw, CBD Oil, Choline, Choline Bitartrate, Choline Citrate, Citicoline, CDP-Choline, Centrophenoxine, Coconut & MCT Oil, Coluracetam, CoQ10 & Ubiquinol, Creatine, DHA (Omega 3), DHEA, DMAE, 5-HTP, Forskolin (Coleus root), GABA, Ginkgo Biloba, Ginseng, Gotu Kola, Glycine, Holy Basil (Tulsi), Huperzine-A, Iodine, Kava Kava, Kratom, Lion's Mane, L- Carnosine, L-Dopa (Mucuna Pruriens), Lemon Balm, L-Glutamine, Lithium Orotate, L-Theanine, Maca, Magnesium, Medicinal Mushrooms, Methylene Blue, Melatonin, N-Acetyl L-Cysteine, N-Acetyl L-Tyrosine, NADH, Nefiracetam, Nicotine, Noopept, Oat Straw, Oxiracetam, Phenibut, Phenylpiracetam, Picamilon, Pine Bark Extract, Piperine, Piracetam, Rhodiola Rosea, Phenylalanine, Phenylethylamine (PEA), Phosphatidylcholine (PC), Phosphatidylserine (PS), PQQ, Pramiracetam, Pterostilbene, Quercetin, Resveratrol, Rosemary, Saffron, SAM-e, St John's wort, Sulbutiamine, Taurine, Tryptophan, Turmeric, Tyrosine, Uridine Monophosphate, Valerian, Vinpocetine, Vitamin B1 (Thiamine), Vitamin B3 (Niacin), Vitamin B5 (Pantothenic Acid), Vitamin B6 (Pyridoxine), Vitamin B8 (Inositol), Vitamin B9 (Folate), Vitamin B12 (Cobalamin), Vitamin D, and Zinc.

9. The method of claim 8, wherein the agent is a peptide that comprises the amino acid sequence of MEHFPGP (SEQ ID NO:1) or an analog thereof.

10. The method of claim 9, wherein the analog comprises an amino acid sequence selected from the group consisting of MGHFPGP (SEQ ID NO:3), MEHFXPGP (SEQ ID NO:4), MGHFXPGP (SEQ ID NO:5), MEHFPAP (SEQ ID NO:6), MEHFXPAP (SEQ ID NO:7), and MGHFXPAP (SEQ ID NO:8), wherein X represents any amino acid residue.

11. The method of claim 9, wherein the peptide is N-terminally acetylated.

12. The method of claim 9, wherein the peptide is C-terminally amidated.

13. The method of claim 9, wherein the peptide is 20 amino acid residues or fewer in length.

14. The method of claim 1, wherein the administering is intranasal.

15. A method for treating a neurological mucopolysaccharidosis (MPS) in a patient in need thereof, comprising intranasal administration to the patient an effective amount of a peptide that comprises an amino acid sequence selected from the group consisting of MEHFPGP (SEQ ID NO:1), MGHFPGP (SEQ ID NO:3), MEHFXPGP (SEQ ID NO:4), MGHFXPGP (SEQ ID NO:5), MEHFPAP (SEQ ID NO:6), MEHFXPAP (SEQ ID NO:7), and MGHFXPAP (SEQ IDNO:8), wherein X represents any amino acid residue.

16. The method of claim 15, wherein the patient has decreased biological activity or physiological level of brain-derived neurotrophic factor (BDNF) as compared to a healthy subject.

17. The method of claim 15, wherein the patient has decreased synaptic transmission or decreased physiological level of SYN1, PSD95, VGLUT1, Gephyrin, or VGAT.

18. The method of claim 15, further comprising monitoring the treatment by checking the level of BDNF in the patient.

19. The method of claim 15, wherein the patient suffers from MPS III.

20-26. (canceled)

27. An isolated peptide that comprises an amino acid sequence selected from the group consisting of MEHFPGP (SEQ ID NO:1), MGHFPGP (SEQ ID NO:3), MEHFXPGP (SEQ ID NO:4), MGHFXPGP (SEQ ID NO:5), MEHFPAP (SEQ ID NO:6), MEHFXPAP (SEQ ID NO:7), and MGHFXPAP (SEQ ID NO:8), wherein X represents any amino acid residue, wherein the peptide is N-terminal acetylated and/or C-terminal amidated.


SEQUENCE LISTING (8 sequences)

SEQ ID NO: 1 - protein, 7 aa, Artificial Sequence
  Synthetic
MEHFPGP

SEQ ID NO: 2 - protein, 267 aa, Artificial Sequence
  Synthetic
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 3 - protein, 7 aa, Artificial Sequence
  Synthetic
MGHFPGP

SEQ ID NO: 4 - protein, 8 aa, Artificial Sequence
  Synthetic
  MISC_FEATURE (5)..(5): Xaa is any amino acid
MEHFXPGP

SEQ ID NO: 5 - protein, 8 aa, Artificial Sequence
  Synthetic
  MISC_FEATURE (5)..(5): Xaa is any amino acid
MGHFXPGP

SEQ ID NO: 6 - protein, 7 aa, Artificial Sequence
  Synthetic
MEHFPAP

SEQ ID NO: 7 - protein, 8 aa, Artificial Sequence
  Synthetic
  MISC_FEATURE (5)..(5): Xaa is any amino acid
MEHFXPAP

SEQ ID NO: 8 - protein, 8 aa, Artificial Sequence
  Synthetic
  MISC_FEATURE (5)..(5): Xaa is any amino acid
MGHFXPAP